(19) United States
(12) Patent Application Publication
(10) Pub. No.: US 2016/0074421 A1
(43) Pub. Date: Mar. 17, 2016

(54) METHOD FOR THE TREATMENT OF DISEASES CAUSED BY HAEMORRHAGIC VIRUSES AND COMPOUNDS FOR USE IN SAID TREATMENT

(71) Applicants: MATTI SIRÈN, Helsingfors (FI); Pontus GALLEN-KALLELA SIRÈN, Helsingfors (FI)

(72) Inventors: MATTI SIRÈN, Helsingfors (FI); Pontus GALLEN-KALLELA SIRÈN, Helsingfors (FI)

(21) Appl. No.: 14/850,988

(22) Filed: Sep. 11, 2015

(30) Foreign Application Priority Data
Sep. 12, 2014 (SE) 1451057-2

(51) Int. Cl. A61K 31/6615; C07F 9/06; A23L 1/29; A61K 45/06

(52) U.S. Cl. CPC A61K 31/6615; A61K 45/06; C07F 9/062; A23L 1/296; A23V 2002/00

(57) ABSTRACT

Diseases caused by haemorrhagic viruses, such as the Ebola and Marburg viruses, can be treated by the administration of compounds comprising a high density negatively charged domain of vicinally oriented radicals. A method of treatment, a pharmaceutical composition, and a nutritional composition are disclosed.

### TECHNICAL FIELD

The present description relates to the treatment of diseases caused by filoviruses (members of the family Filoviridae) also called haemorrhagic viruses, such as for example the Ebola and Marburg viruses. A method for the treatment of such diseases, as well as compounds for use in said method, is disclosed.

### BACKGROUND

Outbreaks caused by haemorrhagic viruses, belonging to the family Filoviridae, represent a major public health issue in sub-Saharan Africa. Filoviridae is a taxonomic group of enveloped, non-segmented, negative-strand RNA viruses that include the genera Marburg virus and Cueva virus, with a single species each, and Ebola virus, with five distinct species.

The perhaps most notorious of the above, the ebolavirus, is associated with a case fatality rate of 30 to 90%, depending on the virus species. The fatality rate is usually less than 40% for Bundibugyo ebolavirus, approximately 50% for Sudan ebolavirus, and ranging from 70-90% for Zaire ebolavirus (Feldman et al., Ebola hemorrhagic fever, Lancet, 2011; 377:849-62).

Three ebolavirus species have caused large outbreaks in sub-Saharan Africa: Zaire ebolavirus (EBOV), Sudan ebolavirus, and the recently described Bundibugyo ebolavirus. Epidemics have occurred in the Democratic Republic of Congo, Sudan, Gabon, Republic of Congo, and Uganda (S. Baize et al., Emergence of Zaire Ebola Virus Disease in Guinea—Preliminary Report, New England Journal of Medicine, published at NEJM.org on Apr. 16, 2014, DOI: 10.1056/NEJMoa1404505).

Ebolavirus disease manifests itself through initial nonspecific signs and symptoms such as fever and malaise, followed by anorexia, headache, myalgia, arthralgia, sore throat, chest or retrosternal pain, conjunctival infection, lumbosacral pain and maculopapular rash. Therefore, early-stage ebolavirus infection may be confused with other infectious diseases, such as malaria, typhoid fever, septicemia including meningococcemia, and pneumonia. Later symptoms include nausea, vomiting, epigastric and abdominal pain and diarrhea. Hemorrhage is seen in about 50% of patients, mostly in later stages, usually leading to death within days. Patients die from complications such as multiorgan failure or septic shock (Briand et al., The International Ebola Emergency, New England Journal of Medicine, DOI: 10:1056/NEJMp1409858)

Specific conditions in hospitals and communities in Africa facilitate the spread of the disease from human to human. The most lethal route of spread is through parenteral infections. In Africa, many patients, including those infected with filoviruses, receive injections of various medications at health centers or from pharmacists or healers, and then unsterilized needles and syringes are reused, sometimes to administer medicines drawn from multidose vials. The single-use injection equipment that has been such a boon to the developed world has spelled disaster for the poorest nations. Plastic syringes cannot be heat sterilized and are usually simply rinsed. Needles are usually not sterilized, either. Even when syringes and needles designed for reuse are available, it is common for hospitals to own only a handful of injection sets, which tend to be reused without sterilization simply for logistic reasons. Indeed, it is a poorly kept secret that such reuse is common practice in African countries, where it has been implicated in the spread of Hepatitis C and, almost certainly, human immunodeficiency virus (HIV). Equally important in spreading filoviruses in hospitals is the inadequate use of barriers by nurses. Gloves are used only rarely—often not even during surgical procedures—and gowns are not commonly available. In many places, patients are cared for at home, and infect their care provides. In addition, extensive viral involvement of the subcutaneous tissues brings the virus very close to anyone who cares for patients or prepares their bodies for burial. In well-equipped modern hospitals, the incidence of transmission is very low, primarily because unprotected contact with the patients and their blood rarely occurs (C. J. Peters, Marburg and Ebola—Arming ourselves against the deadly filoviruses, New England Journal of Medicine, 352; 25, Jun. 23, 2005, 2571-2573).

To date, more than 1000 people, including numerous health care works, have been killed by ebolavirus disease in 2014, and the number of cases in the current outbreak now exceeds the number of all previous outbreaks combined. Indirect effects include disruption of standard medical care, including for common and deadly conditions such as malaria, and substantial economic losses, insecurity, and social disruption in countries that were already struggling to recover from decades of war (Frieden et al., Ebola 2014—New Challenges, New Global Response and Responsibility, New England Journal of Medicine, DOI: 10.1056/NEJMp1409903)

The infection mechanism or mode of action of the hemorrhagic viruses is not entirely clarified. The nature of the virus, the rapid progression of the disease, and the dangers associated with handling the virus and infected samples, makes scientific studies complicated. It is speculated that, following entry into the body, the virus attacks macrophages and monocytes, relying upon host antibodies and complement component 1 for efficient infection. The white blood cells respond by releasing large amounts of pro-inflammatory cytokines that increase the permeability of the vascular endothelium, which facilitates easier entry into the virus's secondary targets, endothelial cells. These cytokines also recruit more macrophages to the area, maximizing the number of cells that the Ebola virus can use to spread throughout the body. In the meantime, hepatocytes are being destroyed by the virus, ensuring that these cell signals cannot be cleared from the bloodstream, resulting in an accumulation of cytokines in the bloodstream.

Following glycosylphosphatidylinositol-mediated receptor binding, Ebola virions are taken into endothelial cells via macropinocytosis. After their formation, macropinosomes move further into the cytoplasm to acquire new markers or fuse with other vesicles of the standard endolysosomal pathway. This eventually moves the Ebola virions to more acidic compartments such as early and late endosomes that assist in the pH dependent fusion of viral and cellular membranes. During this process, the cell detaches from its neighbors and loses contact with its basement membrane thanks to a mechanism of glycan mediated steric occlusion by glycosylphosphatidylinositol. The newly created particles then leave via lipid rafts, leaving a destabilized vascular system responsible for the massive blood loss characteristic of Ebola patients.

Meanwhile, the immune system becomes more and more unbalanced. Interferons are not being made because the viral proteins interfere with nearly every step in the process. White blood cells are trapped inside the circulatory system because sGP limits their movement. Macrophages and monocytes are releasing a cocktail of pro-inflammatory cytokines that destroy the vascular endothelium, but also activate the coagulation cascade. This puts the patient's body in a paradoxical state which can result in death from hypovolemic shock from massive hemorrhage, or from catastrophic thrombosis, the formation of blood clots around the body.

However, to sum up, the information regarding the mode of action at molecular level beyond the primary interactions between the virus agent and the target cells remains poor.

Research and development efforts in order to find a therapeutic drug molecule to combat the ebolavirus infection have so far not been successful. One reason is probably the enigmatic character of the ebolavirus. Another serious obstacle resides in the extremely demanding and rigorous security requirements governing all handling of the virus.

Until today, the use of the RNA interference (RNAi) is the only treatment for the ebolavirus disease having advanced to the stage of human clinical trials. This approach involves using RNA molecules to inhibit protein synthesis, and thus prevent the virus from replicating.

TKM-Ebola (Tekmira Pharmaceuticals Corp.) is an experimental drug based on RNAi. In a 2010 study, researchers tested the drug on four monkeys, giving them seven doses of the medicine after they were infected with high doses of Ebola. The drug worked to protect them from the disease.

ZMapp (Mapp Biopharmaceutical Inc) is another experimental biopharmaceutical drug comprising three humanized monoclonal antibodies under development as a treatment for Ebola virus disease. In animal tests it showed promising results by saving eighteen monkeys who were given lethal doses of the Ebola virus. The drug has been tested in humans during the 2014 West Africa Ebola virus outbreak and has been credited as helping to save lives, but it has not been subjected to a randomized clinical trial to prove its safety or its efficacy.

It can be speculated if the blend of three different antibodies is not based on the fact that a single target/receptor molecule of the Ebola virus has still not been found.

The eradication of the disease would however require the development of a safe vaccine against the virus, a task complicated by the fact that the filoviridae viruses, such as Ebola and Marburg viruses seem to be capable of rapid and potent suppression of innate antiviral immune responses.

While there is an ongoing development of antiviral treatments and vaccines, there remains a need for new strategies for the treatment, alleviation and possibly also prevention of infections caused by filoviridae, such as Ebola and Marburg viruses. To the best knowledge of the inventors, there is currently no practically useful and safe therapeutic agent for the treatment of the hemorrhagic fever syndrome. As evident for anyone following the news coverage of the raging epidemic, the need for improved treatment is indeed acute.

An object of the present disclosure is thus to provide an alternative method of preventing, alleviating and/or treating filovirus infections, in particular one that is superior, at least in some respect, to methods known in the art.

Another object of the present disclosure is to provide a corresponding means, i.e. suitable compounds and pharmaceutical compositions comprising these compounds.

Further objects of the will become evident from the study of the following summary, the description of preferred embodiments thereof, and the appended claims.

### SUMMARY

The present description relates to the use of a compound comprising a high density, negatively charged domain of vicinally oriented radicals for the preparation of a medicament for preventing, alleviating and/or treating filoviridae infections in a mammal, such as Ebola and/or Marburg virus infections. Preferably the negatively charged domain comprises three or more vicinal phosphorus-containing radicals.

Another embodiment relates to a method of treatment of filoviridae infections in a mammal, such as Ebola and/or Marburg virus infections, comprising the administration of a pharmacologically effective amount of a compound, said compound comprising a high density, negatively charged domain of vicinally oriented radicals.

Further embodiments are disclosed in the following description, examples, listing of embodiments of the invention, and appended claims, all incorporated herein by reference.

Advantages of the these compounds and methods, compared for example to vaccine and RNA based treatments, include that the treatment can be initiated post infection, that the compound or compounds are non-toxic or have low toxicity at the required doses, that there are only few or no side-effects, and that the compound can be produced by chemical synthesis, in large quantities and at a reasonable cost.

### DESCRIPTION

Before the present embodiments are described, it is to be understood that the terminology employed herein is used for the purpose of describing particular embodiments only and is not intended to be limiting, since the scope of the present disclosure will be limited only by the appended claims and equivalents thereof.

It must be noted that, as used in this specification and the appended claims, the singular forms “a,” “an,” and “the” include plural referents unless the context clearly dictates otherwise.

Also, the term “about” is used to indicate a deviation of +/−2% of the given value, preferably +/−5%, and most preferably +/−10% of the numeric values, where applicable.

In particular, the present disclosure relates to the treatment of filoviridae infections in a mammal, such as Ebola and/or Marburg virus infections.

In the context of the present description and claims the terms “Ebola virus” and “ebolavirus” are used interchangeable.

In the context of the present description the term “high density” relates to a domain where there is at least two negative charges being distributed among at least two radicals which are connected with covalent bonds to the carbon skeleton.

In the context of the present description the term “vicinally oriented” relates to radicals being connected to the carbon skeleton to carbon atoms adjacent to each other.

In the context of the present disclosure the term “radical” relates to a chemical group connected with covalent bonds to the carbon skeleton.

Where not otherwise indicated, the doses are given as mg/kg meaning mg drug per kg body weight of the mammal—including man—to be treated.

Without wishing to be bound to any specific theory, the present inventor has observed that diseases caused by filoviridae infections are characterized by a rapidly progressing febrile illness with capillary leakage often resulting in extensive muscular subcutaneous haemorrhage. The illness very often progresses to a chock-like syndrome with multi-organ failure culminating in death. A massive outpour of inflammatory cytokine mediators is part of the clinical picture.

During studies of a parallel phenomenon of disseminated diathesis in animal experiments, the present inventor focused on the disruption of gap junction molecular structures. It was possible to identify a progressive deterioration of the structure and function of ordinary anatomical structures in epithelial and endothelial tissues. Surprisingly, the sodium salt of D-myo-inositol-1,2,6-trisphosphate, administered at non-toxic concentrations, effectively and prevented this deterioration.

This observation was confirmed in animal experiments where the sodium salt of D-myo-inositol-1,2,6-trisphosphate was administered as an intravenous bolus dose, followed by infusion over three hours. A bolus dose of about 17 to about 40 μM/kg body weight followed by a constant rate i.v. infusion of about 40-about 60 μM/kg/h. It is contemplated that lower or higher doses can be used. A skilled person, such as a treating physician, or a research team developing a drug against haemorrhagic viruses can titrate a dose during ongoing treatment, or develop a suitable dose regimen based on studies in vitro and in vivo.

In addition to primates, the Ebola virus can also infect rodents, mice, guinea pigs and also bats, at least under experimental conditions, but the infection is usually not lethal for these animals unless the virus is adapted or enriched, as described in this description, in the context of animal experiments. Interestingly, lymphoid cells do not appear to be susceptible to filovirus infection.

There is a group of proteins called the Tyro 3 family. These molecules are usually widely distributed in many types of cells in mammals, except for lymphocytes and granulocytes. The Tyro 3 family includes a receptor-type tyrosine kinase. It also includes the AXL protein, which has been found to be activated by pseudotypes of filovirus in in vitro studies.

A very interesting finding is that Gas6 activity results in the Tyro 3 proteins moving towards the lysosomes in the cells. At the same time, it has been observed that some interleukins mediate the release of lysosomal Zn into the cytoplasm. These interleukin reactions trigger then activation of three major metabolic pathways; ERK1/2, JAK and P13K/AKT. At least AKT phosphorylation can be abrogated by a Zn²⁺ chelator permeable into the cells.

We as well as others have shown, that zinc ions inhibit the activity of PTEN, the tyrosine phosphatase enzyme. PTEN is a member of a family of protein tyrosine phosphatases, most of which have been shown to be inhibited already by a low concentration of zinc ions.

It is most plausible that for example PTEN exist in its active state free of Zn²⁺ in cells but that stimulation with interleukins, e.g. interleukin-2, leads to an inactivation or inhibition of PTEN activity which gives a free way to the metabolism of tyrosine kinase activity.

Thus, the regulation of tyrosine phosphatase, for example by PTEN, is important for counterbalancing the dysbalance due to over-activity of tyrosine kinase. This can be achieved ty blocking the action of free Zn ions through a chelating reaction outside the cell membrane.

By chelating Zn ions outside the cells, it is possible to balance the tight junction opening which follows when tyrosine phosphatase enzymes are blocked by Zn leading to local over-activity of tyrosine kinases influencing the endothelial tissue and endothelial cells.

Again without wishing to be bound to any specific theory, the present inventor contemplates that the cytokines released in response to virus infection upset the balance governing the tight junction between cells in different tissues, resulting in an escalating dysequilibrium. The cytokines appear to target the vascular system, specific anatomic sites of endothelial cells, apical sites, resulting in dissociation of endothelial cell-to-cell contact manifested as tight junction opening.

The tight junction is believed to be regulated by a delicate balance between kinase enzymes and phosphatase enzymes, which phospholysate and dephospholysate serine, threonine and tyrosine, respectively.

It appears that in particular the tyrosine kinase activation is an important enzyme activity which starts the reaction to open tight junction. If and when the balancing reaction of the tyrosine dephosphorylysation is disturbed, leakage occurs.

Recent research results indicate that the Zn²⁺ ion is a very effective inhibitor of tyrosine phosphatase both in vitro and in vivo. This may be one of the most fundamental cell metabolism reactions in protein activity chemistry.

Addressing this disequilibrium through RNA or antibody based therapies does not seem to be a practical route. In order to be able to block the many different ligand target protein molecules which are activated via phosphorylation, through kinase reactions, a large number of specific antibodies would have to be administered simultaneously. Antibodies would have to be raised against various target molecules, such as receptor molecules, enzymes or the like. to be used in therapy at the same

Instead, the present inventor has realized that the most effective way is to influence specifically the tyrosine phosphorylation dysequilibrium via inhibition of blocking tyrosine phosphatase inactivity via chelating Zn²⁺. This has the capacity to block tyrosine phosphatase activity already at a low nanomolar concentration.

According to the present disclosure it has surprisingly been possible to use a pharmacologically effective amount of a compound comprising a high density, negatively charged domain of vicinally oriented radicals for the preparation of a medicament for preventing, alleviating and/or treating filovirus infections in mammals including man. Preferably the domain is at least doubly negatively charged, the two or more charges being distributed between at least two of the radicals. According to a preferred embodiment of the disclosure the negatively charged domain is capable of complexing divalent cations, such as cadmium, calcium, copper and, in particular, zinc.

The present disclosure also relates to the use of a pharmacologically effective amount of a compound comprising a high density, negatively charged domain of vicinally oriented radicals for preventing, alleviating and/or treating filovirus infections in mammals including man.

According to the present disclosure it is additionally disclosed a method of treatment of filovirus infections, in a patient in need of such treatment wherein a pharmacologically effective amount of a compound comprising a high density, negatively charged domain of vicinally oriented radicals is administered.

The present disclosure also relates to the use of a pharmacologically effective amount of a compound comprising a high density, negatively charged domain of vicinally oriented radicals for preventing, alleviating and/or treating filovirus infections in mammals including man. In the context of the present disclosure is has also surprisingly been found that a compound comprising a high density, negatively charged domain of vicinally oriented radicals can be used for decreasing PIF and AngII induced chymotrypsin-like enzyme activity and for preventing, alleviating and/or treating conditions associated with such enzyme activity.

According to the present disclosure it is further presented a method of inhibiting protein degradation and stimulating protein synthesis in a patient in need of such treatment wherein a pharmacologically effective amount of a compound comprising a high density, negatively charged domain of vicinally oriented radicals is administered.

Different embodiments will now be further described by the detailed disclosure of the compound and uses thereof.

In a preferred embodiment, the negatively charged domain of the compound to be used/administered comprises three or more vicinal phosphorus-containing radicals.

According to a preferred embodiment, a phosphorus-containing radical is one of the general formula I

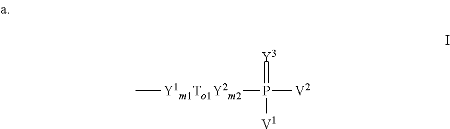

b. or the general formula II

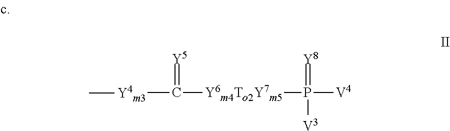

d. wherein
e. V¹ to V⁴ are Y⁹ₘ₆Tₒ₃U
f. Tₒ₁ to Tₒ₃ are (CH₂)ₙ, CH═CH, or CH₂CH═CHCH₂
g. o1 to o3 are 0 to 1
h. n is 0 to 4
i. U is R¹Y¹⁰ₘ₇, CY¹¹Y¹²R², SY¹³Y¹⁴Y¹⁵R³, PY¹⁶Y¹⁷Y¹⁸R⁴R⁵, Y¹⁹PY²⁰Y²¹Y²²R⁶R⁷, CH₂NO₂, NHSO₂R⁸ or NHCY²³Y²⁴R⁹
j. m1 to m7 are 0 to 1
k. Y¹ to Y²⁴ are NHR¹⁰, NOR¹¹, O or S
l. and where R¹ to R¹¹ are
i) hydrogen;
ii) a straight or branched saturated or unsaturated alkyl residue of 1-22 carbon atoms;
iii) a saturated or unsaturated aromatic or non-aromatic homo- or heterocyclic residue of 3-22 carbon atoms and 0-5 hetero atoms selected from nitrogen, oxygen and sulphur;
iv) a straight or branched saturated or unsaturated alkyl residue of 1-22 carbon atoms comprising a saturated or unsaturated aromatic or non-aromatic homo- or heterocyclic substituent of 3-22 carbon atoms and 0-5 hetero atoms selected from nitrogen, oxygen and sulphur;
v) an aromatic or non-aromatic homo- or heterocyclic residue of 3-22 carbon atoms and 0-5 heteroatoms selected from nitrogen, oxygen and sulphur, comprising a straight or branched saturated or unsaturated alkyl substituent of 1-22 carbon atoms.

It is preferred that one or several of the one or more residues and/or substituents of R¹ to R¹¹, groups ii)-v), is/are substituted with from 1 to 6 of hydroxy, alkoxy, aryloxy, acyloxy, carboxy, alkoxycarbonyl, alkoxycarbonyloxy, aryloxycarbonyl, aryloxycarbonyloxy, carbamoyl, fluoro, chloro, bromo, azido, cyano, oxo, oxa, amino, imino, alkylamino, arylamino, acylamino, arylazo, nitro, alkylthio, alkylsulfonyl.

It is preferred that one or several of the one or more straight or branched saturated or unsaturated alkyl residues in R¹ to R¹¹, groups ii), iv), v), is/are chosen from methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, heneicosyl, doeicosyl, isopropyl, isobutyl, isopentyl, isohexyl, isoheptyl, isooctyl, isononyl, isodecyl, isodoecosyl, 2-butyl, 2-pentyl, 2-hexyl, 2-heptyl, 2-octyl, 2-nonyl, 2-decyl, 2-doeicosyl, 2-methylbutyl, 2-methylpentyl, 2-methylhexyl, 2-methylheptyl, 2-methyloctyl, 2-methylnonyl, 2-methyldecyl, 2-methyleicosyl, 2-ethylbutyl, 2-ethylpentyl, 2-ethylhexyl, 2-ethylheptyl, 2-ethyloctyl, 2-ethylnonyl, 2-ethyldecyl, 2-ethyleicosyl, tert-butyl, ethenyl, propenyl, butenyl, pentenyl, hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, heneicosenyl, doeicosenyl, butadienyl, pentadienyl, hexadienyl, heptadienyl, octadienyl, nonadienyl, decadienyl, doeicodienyl, ethynyl, propynyl, doeicosynyl.

It is preferred for a saturated or unsaturated aromatic or non-aromatic homo- or heterocyclic residue or substituent of R¹ to R¹¹, groups iii)-v), to be selected from cyclopropyl, cyclobutyl, cyclopentyl, cyclohexyl, cycloheptyl, cyclooctyl, cyclononyl, cyclodecyl, cycloundecyl, cyclododecyl, cycloridecyl, cyclotetradecyl, cyclopentadecyl, cyclohexadecyl, cycloheptadecyl, cyclooctadecyl, cyclononadecyl, cycloeicosyl, cycloheneicosyl, cyclodoeicosyl, adamantyl, cyclopropenyl, cyclobutenyl, cyclopentenyl, cyclohexenyl, cycloheptenyl, cyclooctenyl, cyclononenyl, cyclodecenyl, phenyl, biphenyl, naphthyl, hydroxyphenyl, aminophenyl, mercaptophenyl, fluorophenyl, chlorophenyl, azidophenyl, cyanophenyl, carboxyphenyl, alkoxyphenyl, acyloxyphenyl, acylphenyl, oxiranyl, thiiranyl, aziridinyl, oxetanyl, thietanyl, azetidinyl, tetrahydrofuranyl, tetrahydrothiophenyl, pyrrolidinyl, tetrahydropyranyl, tetrahydrothiopyranyl, piperidinyl, quinuclidinyl, dioxanyl, dithianyl, trioxanyl, furyl, pyrrolyl, thienyl, pyridyl, quinolyl, benzofuryl, indolyl, benzothienyl, oxazolyl, imidazolyl, thiazolyl, pyridazinyl, pyrimidyl, pyrazinyl, purinyl, carbohydrate.

According to a first particularly preferred embodiment, a phosphorus-containing radical is one of the general formula III

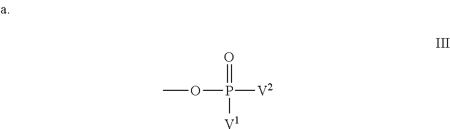

wherein V¹ and V² are, independent of each other, selected from OH, (CH₂)ₚOH, COOH, CONH₂, CONOH, (CH₂)ₚCOOH, (CH₂)ₚCONH₂, (CH₂)ₚCONOH, (CH₂)ₚSO₃H, (CH₂)ₚSO₃, NH₂, (CH₂)ₚNO₂, (CH₂)ₚPO₃H₂, O(CH₂)ₚOH, O(CH₂)ₚCOOH, O(CH₂)ₚCONH₂, O(CH₂)ₚCONOH, (CH₂)ₚSO₃H, O(CH₂)ₚSO₃NH₂, O(CH₂)ₚNO₂, O(CH₂)ₚPO₃H₂, CF₂COOH and p is 1 to 4. In this embodiment, the phosphorus-containing radical is a phosphonate, phosphinate or phosphate including a derivative thereof.

According to this embodiment the domain of high density negatively charged vicinally oriented radicals is linked to a cyclic moiety. The cyclic moiety comprises or consists of a saturated or unsaturated aromatic or non-aromatic homo- or heterocyclic ring. When the moiety comprises a heterocyclic ring the heteroatom(s) thereof are selected from oxygen, nitrogen, sulphur and selenium.

Preferably the cyclic moiety comprises from 4 to 24 atoms, more preferred from 5 to 18 atoms, most preferred 6 atoms. The cyclic moiety is preferably selected from cyclopentane, cyclohexane, cycloheptane, cyclooctane, inositol, monosaccharide, disaccharide, trisaccharide, tetrasaccharide, arabinitol, piperidine, tetra-hydrothiopyran, 5-oxotetrahydrothiopyran, 5,5-dioxotetrahydro-thiopyran, tetrahydroselenopyran, tetrahydrofuran, pyrrolidine, tetrahydrothiophene, 5-oxotetrahydrothiophene, 5,5-dioxotetrahydrothiophene, tetrahydroselenophene, benzene, cumene, mesitylene, naphthalene and phenanthrene. Most preferably the cyclic moiety is selected from the group consisting of inositol, monosacharide, disaccharide, trisaccharide, and tetrasaccharide.

Preferred compounds are such, where the cyclic moiety is a phosphate, a phosphonate or a phosphinate of cyclohexane are in particular 1,2,3-β-cyclohexane-1,2,3-trioltrisphosphate.

When the cyclic moiety is inositol, which is particularly preferred, it is preferably selected from allo-inositol, cis-inositol, epi-inositol, D/L-chiro-inositol, scylloinositol, myoinositol, mucoinositol and neoinositol.

The inositol is preferably a phosphate, a phosphonate, a phosphinate or derivative thereof. Preferably the number of phosphate, phosphonate or phosphinate radicals per inositol moiety is three or more.

Preferred inositols according to this embodiment are selected from the group consisting of inositol-trisphosphate, inositol-tris(carboxymetyl-phosphate), inositol-tris(carbomethylphosphonate), inositol-tris(hydroxymethylphosphonate), tri-O-methyl-inositol-trisphosphate, tri-O-hexyl-inositol-trisphosphate, tri-O-butyl-inositol-trisphosphate, tri-O-pentyl-inositol-trisphosphate, tri-O-isobutyl-inositol-trisphosphate, tri-O-propyl-inositol-trisphosphate, tri-O-(6-hydroxy-4-oxa)hexyl-inositol-trisphosphate, tri-O-3-(ethylsulfonyl)propyl-inositol-trisphosphate, tri-O-3-hydroxypropyl-inositol-trisphosphate, tri-O-(6-hydroxy)-hexyl-inositol-trisphosphate, tri-O-phenylcarbamoyl-inositol-trisphosphate, tri-O-propyl-inositol-tris(carboxymethylphosphate), tri-O-butyl-inositol-tris(carboxymethylphosphate), tri-O-isobutyl-inositol-tris(carboxymethyl-phosphate), tri-O-pentyl-inositol-tris(carboxymethylphosphate), tri-O-hexyl-inositol-tris(carboxymethylphosphate), tri-O-propyl-inositol-tris(carboxymethylphosphonate), tri-O-butyl-inositol-tris(carboxymethyl-phosphonate), tri-O-isobutyl-inositol-tris(carboxymethylphosphonate), tri-O-pentyl-inositol-tris(carboxymethylphosphonate), tri-O-hexyl-inositol-tris(carboxymethylphosphonate), tri-O-propyl-inositol-tris(hydroxymethyl-phosphonate), tri-O-butyl-inositol-tris(hydroxymethylphosphonate), tri-O-isobutyl-inositol-tris(hydroxymethylphosphonate), tri-O-pentyl-inositol-tris(hydroxymethylphosphonate), and tri-O-hexyl-myo-inositol-tris(hydroxymethyl-phosphonate).

If the inositol is a myo-inositol, preferred compounds are selected from the group consisting of D-myo-inositol-1,2,6-trisphosphate, D-myo-inositol-1,2,6-tris(carboxymetyl-phosphate), D-myo-inositol-1,2,6-tris(carbomethylphosphonate), D-myo-inositol-1,2,6-tris(hydroxymethylphosphonate), D-3,4,5-tri-O-methyl-myo-inositol-1,2,6-trisphosphate, D-3,4,5-tri-O-hexyl-myo-inositol-1,2,6-trisphosphate, D-3,4,5-tri-O-butyl-myo-inositol-1,2,6-trisphosphate, D-3,4,5-tri-O-pentyl-myo-inositol-1,2,6-trisphosphate, D-3,4,5-tri-O-isobutyl-myo-inositol-1,2,6-trisphosphate, D-3,4,5-tri-O-propyl-myo-inositol-1,2,6-trisphosphate, D-3,4,5-tri-O-(6-hydroxy-4-oxa)hexyl-myo-inositol-1,2,6-trisphosphate, D-3,4,5-tri-O-3-(ethylsulfonyl)propyl-myo-inositol-1,2,6-trisphosphate, D-3,4,5-tri-O-3-hydroxypropyl-myo-inositol-1,2,6-trisphosphate, D-3,4,5-tri-O-(6-hydroxy)-hexyl-myo-inositol-1,2,6-trisphosphate, D-5-O-hexyl-myo-inositol-1,2,6-trisphosphate, D-3,4,5-tri-O-phenylcarbamoyl-myo-inositol-1,2,6-trisphosphate, D-3,4,5-tri-O-propyl-myo-inositol-1,2,6-tris(carboxymethylphosphate), D-3,4,5-tri-O-butyl-myo-inositol-1,2,6-tris(carboxymethylphosphate), D-3,4,5-tri-O-isobutyl-myo-inositol-1,2,6-tris(carboxymethyl-phosphate), D-3,4,5-tri-O-pentyl-myo-inositol-1,2,6-tris(carboxymethylphosphate), D-3,4,5-tri-O-hexyl-myo-inositol-1,2,6-tris(carboxymethylphosphate), D-3,4,5-tri-O-propyl-myo-inositol-1,2,6-tris(carboxymethylphosphonate), D-3,4,5-tri-O-butyl-myo-inositol-1,2,6-tris(carboxymethyl-phosphonate), D-3,4,5-tri-O-isobutyl-myo-inositol-1,2,6-tris(carboxymethylphosphonate), D-3,4,5-tri-O-pentyl-myo-inositol-1,2,6-tris(carboxymethylphosphonate), D-3,4,5-tri-O-hexyl-myo-inositol-1,2,6-tris(carboxymethylphosphonate), D-3,4,5-tri-O-propyl-myo-inositol-1,2,6-tris(hydroxymethyl-phosphonate), D-3,4,5-tri-O-butyl-myo-inositol-1,2,6-tris(hydroxymethylphosphonate), D-3,4,5-tri-O-isobutyl-myo-inositol-1,2,6-tris(hydroxymethylphosphonate), D,-3,4,5-tri-O-pentyl-myo-inositol-1,2,6-tris(hydroxymethylphosphonate), D-3,4,5-tri-O-hexyl-myo-inositol-1,2,6-tris(hydroxymethyl-phosphonate), D-3,4,5-tri-O-propanoyl-myo-inositol-1,2,6-tris(carboxymethylphosphate), D-3,4,5-tri-O-butanoyl-myo-inositol-1,2,6-tris(carboxymethylphosphate), D-3,4,5-tri-O-isobutanoyl-myo-inositol-1,2,6-tris(carboxymethyl-phosphate), D-3,4,5-tri-O-pentanoyl-myo-inositol-1,2,6-tris(carboxymethylphosphate), D-3,4,5-tri-O-hexanoyl-myo-inositol-1,2,6-tris(carboxymethylphosphate), D-3,4,5-tri-O-propanoyl-myo-inositol-1,2,6-tris(carboxymethylphosphonate), D-3,4,5-tri-O-butanoyl-myo-inositol-1,2,6-tris(carboxymethyl-phosphonate), D-3,4,5-tri-O-isobutanoyl-myo-inositol-1,2,6-tris(carboxymethylphosphonate), D-3,4,5-tri-O-pentanoyl-myo-inositol-1,2,6-tris(carboxymethylphosphonate), D-3,4,5-tri-O-hexanoyl-myo-inositol-1,2,6-tris(carboxymethylphosphonate), D-3,4,5-tri-O-propanoyl-myo-inositol-1,2,6-tris(hydroxymethyl-phosphonate), D-3,4,5-tri-O-butanoyl-myo-inositol-1,2,6-tris(hydroxymethylphosphonate), D-3,4,5-tri-O-isobutanoyl-myo-inositol-1,2,6-tris(hydroxymethylphosphonate), D,-3,4,5-tri-O-pentanoyl-myo-inositol-1,2,6-tris(hydroxymethylphosphonate), and D-3,4,5-tri-O-hexanoyl-myo-inositol-1,2,6-tris(hydroxymethyl-phosphonate).

Inositol triphosphates constitute a preferred group of compounds. Within this group, preferred compounds are myo-inositol-1,2,6-trisphosphate and myo-inositol-1,2,3-trisphosphate, in particular in the form of a sodium salt. Particularly, the penta sodium salt of 1,2,6-D-myo inositol trisphosphate (Na₅H 1,2,6-D-myo-inositol trisphosphate), Mg₃ 1,2,6-D-myo-inositol trisphosphate or Ca₃ 1,2,6-D-myo-inositol trisphosphate).

When the cyclic moiety is a saccharide it is preferably selected from D/L-ribose, D/L-arabinose, D/L-xylose, D/L-lyxose, D/L-allose, D/L-altrose, D/L-glucose, D/L-mannose, D/L-gulose, D/L-idose, D/L-galactose, D/L-talose, D/L-ribulose, D/L-xylulose, D/L-psicose, D/L-sorbose, D/L-tagatose, D/L rhamnose and D/L-fructose, including derivatives thereof.

Preferably the compound is a phosphate, a phosphonate or a phosphinate of a saccharide. Preferably the number of phosphate, phosphonate or phosphinate radicals per saccharide moiety is three or more. One or more of the hydroxyl groups on the saccharide moiety not bound to phosphorous can be etherified or esterified. Esterification and etherification is particularly preferred since it increases stability and prolongs half-life of the compound in vivo by reducing susceptibility to enzymatic degradation.

Preferred compounds having a saccharide moiety selected from mannose-2,3,4-trisphosphate, galactose-2,3,4-trisphosphate, fructose-2,3,4-trisphosphate, and altrose-2,3,4-trisphosphate and rhamnose-2,3,4-trisphosphate. Most preferred is to select the compound from R¹-6-O—R²-α-D-manno-pyranoside-2,3,4-trisphosphate, R¹-6-O—R²-α-D-galacto-pyranoside-2,3,4-trisphosphate, R¹-6-O—R²-α-D-altropyranoside-2,3,4-trisphosphate and R¹-6-O—R²-β-D-fructopyranoside-2,3,4-trisphosphate, wherein R¹ and R² independent of each other are defined as above, and preferably are methyl, ethyl, propyl, butyl, pentyl, or hexyl.

Preferred compounds comprise a saccharide moiety in which R¹ and/or R² are substituted in the aforementioned manner are selected from methyl-6-O-butyl-α-D-mannopyranoside-2,3,4-trisphosphate, methyl-6-O-butyl-α-D-galactopyranoside-2,3,4-trisphosphate, methyl-6-O-butyl-α-D-glycopyranoside-2,3,4-trisphosphate, methyl-6-O-butyl-α-D-altropyranoside-2,3,4-trisphosphate, methyl-6-O-butyl-β-D-fructopyranoside-2,3,4-trisphosphate, 1,5-anhydro-D-arabinitol-2,3,4-trisphosphate, 1,5-anhydroxylitol-2,3,4-trisphosphate, 1,2-O-ethylene-β-D-fructopyranoside-2,3,4-trisphosphate, methyl-α-D-rhamno-pyranoside-2,3,4-trisphosphate, methyl-α-D-mannopyranoside-2,3,4-trisphosphate, methyl-6-O-butyl-α-D-mannopyranoside-2,3,4-tris-(carboxymethylphosphate), methyl-6-O-butyl-α-D manno-pyranoside-2,3,4-tris(carboxymethyl-phosphonate), methyl-6-O-butyl-α-D-manno-pyranoside-2,3,4-tris(hydroxymethyl-phosphonate), methyl-6-O-butyl-α-D-galactopyranoside-2,3,4-tris(carboxymethyl-phosphate), methyl-6-O-butyl-α-D-galacto-pyranoside-2,3,4-tris(carboxymethyl-phosphonate), methyl-6-O-butyl-α-D-galacto-pyranoside-2,3,4-tris(hydroxymethyl-phosphonate), methyl-6-O-butyl-α-D-glucopyranoside-2,3,4-tris(carboxymethylphosphate), methyl-6-O-butyl-α-D-glucopyranoside-2,3,4-tris(carboxymethyl-phosphonate), methyl-6-O-butyl-α-D-glucopyranoside-2,3,4-tris(hydroxymethyl-phosphonate), methyl-6-O-butyl-α-D-altropyranoside-2,3,4-tris-(carboxymethylphosphate), methyl-6-O-butyl-α-D-altropyranoside-2,3,4-tris-(carboxymethyl-phosphonate), methyl-6-O-butyl-α-D-altropyranoside-2,3,4-tris-(hydroxymethylphosphonate), methyl-6-O-butyl-β-D-fructo-pyranoside-2,3,4-tris-(carboxymethylphosphate), methyl-6-O-butyl-β-D-fructopyranoside-2,3,4-tris-(carboxymethyl-phosphonate), and methyl-6-O-butyl-β-D-fructo-pyranoside-2,3,4-tris-(hydroxymethylphosphonate).

When the cyclic moiety is an arabinitol, the compound is preferably a phosphate, phosphonate or phosphinate of arabinitol. Preferred arabinitol compounds, comprising a heterocyclic moiety, are selected from 1,5-dideoxy-1,5-iminoarabinitol-2,3,4-trisphosphate,1,5-dideoxy-1,5-iminoarabinitol-2,3,4-tris-(carboxymethylphosphate), 1,5-dideoxy-1,5-imino-arabinitol-2,3,4-tris(carboxymethyl-phosphonate), 1,5-dideoxy-1,5-iminoarabinitol-2,3,4-tris(hydroxymethylphosphonate), 1,5-dideoxy-1,5-imino-N-(2-phenylethyl)arabinitol-2,3,4-trisphosphate, 1,5-dideoxy-1,5-imino-N-(2-phenylethyl)-arabinitol-2,3,4-tris(carboxymethyl-phosphate), 1,5-dideoxy-1,5-imino-N-(2-phenylethyl)arabinitol-2,3,4-tris-(carboxy-methylphosphonate), and 1,5-dideoxy-1,5-imino-N-(2-phenyl-ethyl)arabinitol-2,3,4-tris(hydroxymethylphosphonate).

When the cyclic moiety comprises one or more hydroxyl groups not bound to phosphorous-containing radicals at least one of said hydroxyl groups can be derivatized in the form of an ether or an ester. Esterification and etherification are preferred since there is an increase in stability and prolongation of half-life of this type of compounds in vivo due to reduced susceptibility to enzymatic degradation.

At least one of the hydroxyl groups of the cyclic moiety not bound to phosphorous-containing radicals can be derivatized to form an ester having the general formula IV

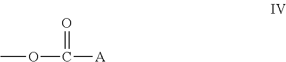

According to a first alternative A is a straight or branched saturated or unsaturated alkyl residue containing 1 to 24 carbon atoms to be selected from the group consisting of methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, heneicosyl, doeicosyl, isopropyl, isobutyl, isopentyl, isohexyl, isoheptyl, isooctyl, isononyl, isodecyl, isodoecosyl, 2-butyl, 2-pentyl, 2-hexyl, 2-heptyl, 2-octyl, 2-nonyl, 2-decyl, 2-doeicosyl, 2-methylbutyl, 2-methylpentyl, 2-methylhexyl, 2-methylheptyl, 2-methyloctyl, 2-methylnonyl, 2-methyldecyl, 2-methyleicosyl, 2-ethylbutyl, 2-ethylpentyl, 2-ethylhexyl, 2-ethylheptyl, 2-ethyloctyl, 2-ethylnonyl, 2-ethyldecyl, 2-ethyleicosyl, tert-butyl, ethenyl, propenyl, butenyl, pentenyl, hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, heneicosenyl, doeicosenyl, butadienyl, pentadienyl, hexadienyl, heptadienyl, octadienyl, nonadienyl, decadienyl, doeicodienyl, ethynyl, propynyl and doeicosynyl.

According to a second alternative A is a saturated or unsaturated aromatic or non-aromatic homo- or heterocyclic residue or substituent to be selected from the group consisting of cyclopropyl, cyclobutyl, cyclopentyl, cyclohexyl, cycloheptyl, cyclooctyl, cyclononyl, cyclodecyl, cycloundecyl, cyclododecyl, cycloridecyl, cyclotetradecyl, cyclopentadecyl, cyclohexadecyl, cycloheptadecyl, cyclooctadecyl, cyclononadecyl, cycloeicosyl, cycloheneicosyl, cyclodoeicosyl, adamantyl, cyclopropenyl, cyclobutenyl, cyclopentenyl, cyclohexenyl, cycloheptenyl, cyclooctenyl, cyclononenyl, cyclodecenyl, phenyl, biphenyl, naphthyl, hydroxyphenyl, aminophenyl, mercaptophenyl, fluorophenyl, chlorophenyl, azidophenyl, cyanophenyl, carboxyphenyl, alkoxyphenyl, acyloxyphenyl, acylphenyl, oxiranyl, thiiranyl, aziridinyl, oxetanyl, thietanyl, azetidinyl, tetrahydrofuranyl, tetrahydrothiophenyl, pyrrolidinyl, tetrahydropyranyl, tetrahydrothiopyranyl, piperidinyl, quinuclidinyl, dioxanyl, dithianyl, trioxanyl, furyl, pyrrolyl, thienyl, pyridyl, quinolyl, benzofuryl, indolyl, benzothienyl, oxazolyl, imidazolyl, thiazolyl, pyridazinyl, pyrimidyl, pyrazinyl, purinyl and carbohydrate.

According to a third alternative A is (CH₂)ₙOR¹² where n is an integer between 1 and 10 and where R¹² is hydrogen, a substituted or unsubstituted straight or branched alkyl, cycloalkyl, aryl or alkaryl; preferably n is between 2 and 4 and R¹² is hydrogen or a lower alkyl such as methyl, ethyl or propyl.

According to a fourth alternative A is (CH₂)ₙZ(CH₂)ₘOR¹² where n and m is an integer between 1 and 10, where Z is oxygen or sulphur and where R¹² is hydrogen, a substituted or unsubstituted straight or branched alkyl, cycloalkyl, aryl or alkaryl; preferably n is 1, m is between 2 and 4 and R¹² is hydrogen or a lower alkyl such as methyl, ethyl or propyl.

According to a fifth alternative A is (CH₂)ₙOCOR¹² where n is an integer between 1 and 10 and where R¹² is hydrogen, a substituted or unsubstituted straight or branched alkyl, cycloalkyl, aryl or alkaryl; preferably n is between 2 and 4 and R¹² is hydrogen or a lower alkyl such as methyl, ethyl or propyl.

According to a sixth alternative A is (CH₂)ₙCOOR¹² where n is an integer between 1 and 10 and where R¹² is hydrogen, a substituted or unsubstituted straight or branched alkyl, cycloalkyl, aryl or alkaryl; preferably n is between 2 and 4 and R¹² is hydrogen or a lower alkyl such as methyl, ethyl or propyl.

According to a seventh alternative A is (CH₂)ₙ OCOOR¹² where n is an integer between 1 and 10 and where R¹² is hydrogen, a substituted or unsubstituted straight or branched alkyl, cycloalkyl, aryl or alkaryl; preferably n is between 2 and 4 and R¹² is hydrogen or a lower alkyl such as methyl, ethyl or propyl.

According to an eight alternative A is (CH₂)ₙR¹² where n is an integer between 1 and 10 and where R¹² is hydrogen, a substituted or unsubstituted straight or branched alkyl, cycloalkyl, aryl or alkaryl; preferably n is between 2 and 4 and R¹² is hydrogen, a lower alkyl such as methyl, ethyl or propyl.

According to a ninth alternative A is (CH₂)ₙ OCONR¹²R¹³ where n is an integer between 1 and 10 and where R¹² and R¹³ are hydrogen, a substituted or unsubstituted straight or branched alkyl, cycloalkyl, aryl or alkaryl; preferably n is between 2 and 4 and R¹² and R¹³ are hydrogen or a lower alkyl such as methyl, ethyl or propyl.

The substituent A could be the same at all of the positions or could have different structures following the above definitions.

When the cyclic moiety is an inositol, triesters of the compounds are preferred. Most preferred compounds are selected from the group consisting of tri-O-hexanoyl-inositol-trisphosphate, tri-O-butanoyl-inositol-trisphosphate, tri-O-pentanoyl-inositol-trisphosphate, tri-O-(4-hydroxy)pentanoyl-inositol-trisphosphate, tri-O-isobutanoyl-inositol-trisphosphate, tri-O-propanoyl-inositol-trisphosphate, tri-O-(6-hydroxy-4-oxa)hexanoyl-inositol-trisphosphate, tri-O-3-(ethylsulfonyl)propanoyl-inositol-trisphosphate, tri-O-3-hydroxypropanoyl-inositol-trisphosphate, tri-O-(6-hydroxy)-hexanoyl-inositol-trisphosphate, tri-O-phenylcarbamoyl-inositol-trisphosphate, tri-O-dodecanoyl-inositol-trisphosphate, tri-O-(2-acetoxy) benzoylcarbamoyl-inositol-trisphosphate, tri-O-butylcarbamoyl-inositol-trisphosphate, tri-O-metylcarbamoyl-inositol-trisphosphate, and tri-O-phenylcarbamoyl-inositol-trisphosphate.

When the cyclic moiety is a myo-inositol, triesters of the compounds are preferred. Most preferred compounds are selected from the group consisting of D-3,4,5-tri-O-hexanoyl-myo-inositol-1,2,6-trisphosphate, D-3,4,5-tri-O-butanoyl-myo-inositol-1,2,6-trisphosphate, D-3,4,5-tri-O-pentanoyl-myo-inositol-1,2,6-trisphosphate, D-3,4,5-tri-O-(4-hydroxy)pentanoyl-myo-inositol-1,2,6-trisphosphate, D-3,4,5-tri-O-isobutanoyl-myo-inositol-1,2,6-trisphosphate, D-3,4,5-tri-O-propanoyl-myo-inositol-1,2,6-trisphosphate, D-3,4,5-tri-0-(6-hydroxy-4-oxa)hexanoyl-myo-inositol-1,2,6-trisphosphate, D-3,4,5-tri-O-3-(ethylsulfonyl)propanoyl-myo-inositol-1,2,6-trisphosphate, D-3,4,5-tri-O-3-hydroxypropanoyl-myo-inositol-1,2,6-trisphosphate, D-3,4,5-tri-O-(6-hydroxy)-hexanoyl-myo-inositol-1,2,6-trisphosphate, D-3,4,5-tri-O-phenylcarbamoyl-myo-inositol-1,2,6-trisphosphate, D-3,4,5-tri-O-dodecanoyl-myo-inositol-1,2,6-trisphosphate, D-3,4,5-tri-O-(2-acetoxy) benzoylcarbamoyl-myo-inositol-1,2,6-trisphosphate, D-3,4,5-tri-O-butylcarbamoyl-myo-inositol-1,2,6-trisphosphate, D-3,4,5-tri-O-metylcarbamoyl-myo-inositol-1,2,6-trisphosphate, and D-3,4,5-tri-O-phenylcarbamoyl-myo-inositol-1,2,6-trisphosphate in particular, in the form of their sodium salts. One preferred triester of the compound is D-3,4,5-tri-O-hexanoyl-myo-inositol-1-2,6-trisphosphate in the form of the penta sodium salt.

1,2,6-D-myo-inositol trisphosphate is formed from phytic acid by controlled enzymatic cleavage. 1,2,6-D-myo-inositol trisphosphate is stable in form of its salts that form aqueous solutions near the neutral point. If not otherwise indicated 1,2,6-D-myo-inositol trisphosphate is presumed to be present in such salt form. 1,2,6-D-myo-inositol trisphosphate in form of its salts and pharmaceutical compositions comprising 1,2,6-D-myo-inositol trisphosphate in form of its salts are disclosed in U.S. Pat. No. 4,777,134 A and U.S. Pat. No. 4,735,936 A, respectively. 1,2,6-D-myo-inositol trisphosphate is disclosed to have preventive effect in cardiovascular disease, cerebral disease, diseases of the respiratory system, diseases related to abnormal hormone release (U.S. Pat. No. 5,128,332 A), and other conditions in which neuropeptide Y is said to be involved. 1,2,6-D-myo-inositol trisphosphate does not pass through the cell membrane.

Pharmaceutically acceptable salts, in particular sodium, potassium, calcium and magnesium salts, of the compounds are also comprised. Particularly preferred is the penta sodium salt of 1,2,6-D-myo-inositol trisphosphate (Na₅H 1,2,6-D-myo-inositol trisphosphate) or other pharmaceutically acceptable salts of 1,2,6-D-myo-inositol trisphosphate, in particular the magnesium salt and the calcium salt.

According to a preferred aspect, one or more, in particular all, of the hydroxyl groups in positions 3, 4, and 5 of 1,2,6-D-myo-inositol trisphosphate are esterified, such as with C₂-C₁₀ carboxylic acid, more preferred with saturated C₂-C₁₀ carboxylic acid, even more preferred with saturated and straight-chain C₂-C₁₀ carboxylic acid, most preferred with butyric acid, valeric acid and, in particular, caproic acid.

A preferred triester of the compound is 1D-3,4,5-trishexanoyl-myo-inositol-1-2,6-trisphosphate, in particular including in form of its penta sodium salt.

A pharmacologically effective amount of the compound is an amount that prevents, dampens and even stops the catabolism, in particular an amount that reduces or stops the rate of loss of lean muscle mass.

The present disclosure also makes available a method for inhibiting protein degradation and stimulating protein synthesis in catabolic patients, in particular severely ill patients suffering from a filovirus infection.

In general, the compound is administered in form of one of its pharmaceutically acceptable salts, in particular its sodium salt. Other compounds are preferably administered in a corresponding manner. In the following, a reference to alpha-trinositol comprises reference to the pharmaceutically acceptable salts of 1,2,6-D-myo-inositol trisphosphate, in particular the pentasodium salt.

Preferably the compound or compounds is/are used in essentially pure form, but its/their use in a purity of 80% or more, preferably of 90% or more, most preferred of 95% or more, is also comprised by this disclosure. Impurities accompanying the inositol trisphosphates used and administered according to this disclosure comprise or substantially consist of other pharmaceutically acceptable inositol phosphates. In particular, if the compound is the pentasodium salt of 1,2,6-D-myo inositol trisphosphate, the impurities comprise or substantially consist of other pharmaceutically acceptable inositol phosphates.

The compounds to be used/administered according to embodiments disclosed herein can for example be administered intravenously. When administered intravenously, a preferred amount of alpha-trinositol is given to an adult person as a bolus injection from about 1 mg/kg body weight to about 100 mg/kg body weight, preferably about 5 mg/kg body weight to about 80 mg/kg body weight, preferably about 10 mg/kg body weight to about 60 mg/kg body weight, more preferably from about 20 mg/kg or about 30 mg/kg to about 50 mg/kg, most preferably about 40 mg/kg body weight.

It is preferred to administer alpha-trinositol intravenously at a rate to maintain the plasma level thereof at or near the maximum plasma level obtained by injecting a bolus of alpha-trinositol of from about 1 mg/kg body weight to about 100 mg/kg body weight, 5 mg/kg body weight to about 80 mg/kg body weight, about 10 mg/kg to about 60 mg/kg, more preferred from about 20 mg/kg or about 30 mg/kg to about 50 mg/kg, most preferably about 40 mg/kg body weight.

Alternatively, the administration of two or more separate intravenous bolus injections over a day spaced by from 1 to 12 hrs of the compound of from about 1 mg/kg body weight to about 100 mg/kg body weight, about 5 mg/kg body weight to about 80 mg/kg body weight, about 10 mg/kg to about 60 mg/kg of the compound is preferred, more preferred of from about 20 mg/kg or about 30 mg/kg to about 50 mg/kg, most preferred of about 40 mg/kg body weight.

Based on earlier studies, it is contemplated that the compound could be administered in the form of at least one bolus injection of about 17 to about 40 μM/kg body weight followed by a constant rate i.v. infusion of about 40 to about 60 μM/kg/h. It is contemplated that lower or higher doses can be used. A skilled person, such as a treating physician, or a research team developing a drug against haemorrhagic viruses can titrate a dose during ongoing treatment, or develop a suitable dose regimen based on studies in vitro and in vivo.

If the compound to be used/administered is an ester as described above, such as an ester of alpha-trinositol, the amount administered is about 0.1 mg/kg body weight to about 20 mg/kg body weight, preferably about 1 mg/kg to about 10 mg/kg and more preferably about 4 mg/kg to about 8 mg/kg.

The administration of alpha-trinositol according to the present disclosure to a patient afflicted with a filoviridae infection, for example a disease caused by Ebola or Marburg virus, or a patient at risk of developing such infection can proceed as long as there is manifest filoviridae infection or a risk of such infection, such as over a period of from one day to a week or two weeks and even for a month of more. Due to the nature of alpha-trinositol such treatment is well tolerated. Preferred administration ranges (mg of compound/kg body weight) for other compounds of the present disclosure can be determined by titration of animal models and/or patients with viral infections.

Alternatively, alpha-trinositol or other compounds according to the present disclosure, including their pharmaceutically acceptable salts, is/are administered subcutaneously or intramuscularly. A skilled person, such as a treating physician, or a research team developing a drug against haemorrhagic viruses can develop modes of administration during ongoing treatment, or based on studies in vitro and in vivo.

It is also within the scope of the present disclosure to provide an adequate plasma level of alpha-trinositol or other compounds according to the present disclosure in a patient by means of an implant, such as an infusion pump, which may be implanted and designed for slow release.

A specific embodiment is a pharmaceutical composition comprising the compounds as described above. The composition can be adapted for intravenous administration, including intravenous bolus injection and intravenous infusion over an extended period of time, such as for hours and even a day or more, comprising a pharmacologically effective amount of alpha-trinositol or other compounds according to the description and an aqueous solvent, in particular saline, and a pharmaceutically acceptable carrier. Preferably such composition is in a closed container and in crystalline or amorphous form, including in form of a cryoprecipitate. The composition can also be dispersed in a stabilizing agent or a mixture of stabilizing agents, in particular in one or more of glucose, mannose, sodium chloride.

According to another preferred embodiment, the composition for intravenous infusion additionally comprises an analgesic agent, in particular an opioid agonist. It is preferred for the opioid agonist to be selected from morphine, nalorphine, nalbuphine, levorphanol, racemorphan, levallorphan, dextromethorphan, cyclorphan, butorphanol, pentazocine, phenazocine, cyclazocine, ketazocine, pethidine, meperidine diphenoxylate, anileridine, piminodine, fentanil, ethoheptazine, alphaprodine, betaprodine, 1-methyl-4-phenyl-1,2,5,6-tetrahydropyridine (MPTP), loperamide, sulfentanil, alfentanil, remifentanil, lofentanil, methadone, d-propoxyphene, isomethandone, levo-alpha-acetylmethadol (LAAM), naloxone, naltrexone, natrindole, oripavine and its derivatives, codeine, heterocodeine, morphinone, dihydromorphine, dihydrocodeine, dihydromorphinone, dihydrocodeinone, 6-desoxumorphine, oxymorphone, oxycodone, 6-methylene-dihydromorphine, hydrocodone, hydromorphone, metopon, apomorphine, normorphine, N-(2-phenylethyl)-normorphine, etorphine, buprenorphine, spiradoline, enadoline or asimadoline.

Further according to a particular embodiment, an effective amount of the previously recited compounds generally described in the foregoing to comprise a high density, negatively charged domain of vicinally oriented radicals, preferably capable of complexing divalent cations can be combined with nutrients with the purpose of treating wasting or other serious forms of catabolism associated with filovirus infections or other conditions which frequently are difficult or impossible to reverse with conventional nutritional regimens. Such catabolic conditions may for example be induced from sepsis and severe burns or other catabolic conditions as specified above.

A therapy including the recited type of compounds can be an adjunct therapy wherein the components are administered separately according to suitable predetermined schemes, or it can be a co-administration in a form suitable or conventional for administering parenteral or enteral nutrients. Numerous products for nutrition in critical care are available and they are commonly based on one or several of lipid emulsions, sources of amino acids and carbohydrates (sugars). Especially for parenteral nutrition, products are developed with special consideration to compatibility of the ingredients during terminal sterilization and long-term storage. The persons skilled in this technology are also aware of nutritional constituents with documented usefulness in catabolic conditions such as omega-3-fatty acids (from an oil source) and branched chain amino acids (e.g. valine, leucine and isoleucine).

The combination therapy with selected nutrients aims at further enhancing the treatment of the severely ill patients by replenishing depleted supplies of nutrients in the skeletal muscles and restore their overall body mass. According to one embodiment, a nutritional composition is provided comprising an inositol triphosphate or an ester thereof, or a mono- or disaccharide having three or more phosphate radicals per saccharide moiety or an ester thereof; and at least one nutrient selected from the group consisting of lipid emulsions, fluid sources of amino acids and carbohydrates.

When the composition is adapted to parenteral administration it comprises suitably manufactured constituents in a vehicle suitable for this administration route. A nutritional composition for oral or enteral administration may include taste enhancers and conventional ingredients well known to practitioners in this field. Examples of suitable nutrients are fluid sources of amino acids or conjugates or precursors thereof (e.g. peptides), lipid emulsions comprising oil phases with long- or medium chain fatty acids and carbohydrate solutions (comprising glucose and/or other energy rich compounds).

The nutrient compositions may further comprise constituents well-known in the field such as vitamins, trace elements, electrolytes, isotonicity adjusters and the like, as well complementary drugs dependent on the clinical situation.

Another embodiment relates to the use of an inositol triphosphate or an ester thereof, or a mono- or disaccharide having three or more phosphate radicals per saccharide moiety or an ester thereof; and at least one nutrient selected from the group consisting of lipid emulsions, fluid sources of amino acids and carbohydrates for the preparation of a nutritional supplement for preventing and/or treating wasting or catabolic conditions associated with filovirus infections.

Preferably, the inositol triphosphate or an ester thereof, or a mono- or disaccharide having three or more phosphate radicals per saccharide moiety or an ester thereof is supplied to a composition adapted for parenteral administration just its before administration. In one embodiment, such composition comprises a solution of carbohydrates.

In normal intake, the use of the nutritional supplement provides about 5 to about 80, preferably about 10 to about 60 mg per kg body weight of inositol triphosphate or an ester thereof, or a mono- or disaccharide having three or phosphate radicals per saccharide moiety or an ester thereof to a patient.

### EXAMPLES

### General Considerations

Different embodiments will now be further disclosed in the following non-limiting examples. In vitro and in vivo studies are planned, with the aim of measuring the reaction activity of tyrosine phosphatase as a function of the progression of Ebola virus infection, as well as in response to the administration of 1,2,6-D-myo inositol trisphosphate (IP3) and isomers and derivatives thereof.

The pathogenesis of Ebola haemorrhagic fever can be studied in cynomolgus primate animal experimental models. It would however be useful to study also the susceptibility of human endothelial cells to Ebola virus infection and disease progression.

The Ebola virus infection causes a very severe and frequently fatal haemorrhagic disease in humans and non-human primates. To find a therapeutic adequately functioning drug, it is important to first clarify the mode of action behind the pathogenesis of the Ebola virus and Ebola haemorrhagic fever. The Ebola haemorrhagic fever has been characterized by hypotension, generalised fluid distribution defects, lymphopenia, coagulation disorders and, during the last phase of disease, also haemorrhage.

Disseminated intravascular coagulation (DIC) is a manifesting, prominent symptom of Ebola virus infection. The multiple organ failure and high mortality rates seem to be caused by coagulation abnormalities, including systemic intravascular coagulation cascade activation. This abnormality leads to widespread deposition of fibrin in the circulation.

The most fatal phenomenon in Ebola virus infection is the vascular injury. But this means not that the injury is the same as endothelial cell injury. The build-up of pathogenesis tells us that it is a question of endothelial tissue dysfunction that is the boiling point of the pathogenesis of haemorrhagic fever. More than 1000 known cases of Ebola infection have been registered until in the beginning of 2000, and many more since then, but very few have been analysed by necropsy.

Regarding the role of the endothelium in the pathogenesis of the disease, Ebola infection of endothelial cells has been well analysed and documented in non-human primate. However, as the experimental animals were euthanized when moribund, the experiments could not shed any light on the pathogenesis of Ebola infection during the most important time period close to death.

Therefore the present inventors set out to examine what really takes place in the endothelial tissue during the last stage of disease. The aim is to develop drugs useful for human use. The hypothesis of the present inventors it that the Ebola virus infection mediates distribution of endothelia via indirect molecular route rather than via direct action, injuring endothelial cells.

One of the most notable findings was that previous studies have shown that Ebola virus infected monkeys did not loose the total serum proteins during the first 3-4 days after infection, whereas the serum albumin levels were found to decrease by day 4. The selective loss of a small molecular weight protein such as albumin was remarkable and indicates that leakage of endothelial tissue can be the most vulnerable event in the pathogenesis of Ebola virus infection in the human body.

An excessive loss of albumin would of course result in a reduced osmotic pressure in plasma. It has also been well documented that TNF-alpha can increase albumin permeation into the systemic vasculature.

The inventors base their theory on that it is possible to find an effective therapeutic drug molecule which is capable of re-balancing the dysbalance/dysequilibrium of the endothelium by inhibiting tyrosine phosphatase enzyme activity by using chelating, non-toxic molecule according to the description, e.g. alpha-trinositol, and thus inhibiting the leakage of the endothelium.

Both in vitro and in vivo experiments are showing this effect.

### In Vitro Experiments

Is it possible to identify the basic reaction on a molecular level and this way resolve the enigmatic findings made during studies of the Ebola virus syndrome and in animal experiments and studies of infected human patients?

Because research using Ebola viruses cannot be performed in normal laboratories, the virus studies and experiments must be made under strict adherence to safety recommendations for the handling of highly infectious materials.

A practical way to initiate Ebola virus studies was therefore the application of findings already made, and studying the already voluminous literature. By reviewing the experimental set-up and results achieved, both animal experiments and in vitro studies, and analysing the metabolism of human and animal tissue and interpreting these results in the light of findings made in the Ebola virus haemorrhagic fever, the present inventors came to surprising conclusions.

This way, it was possible to answer the question if there really is a correlation between albumin levels in plasma, Zn concentration, and the concentration of different immunological markers, and reduction of body weight. Through this indirect approach, the inventors found that:

There is a strong positive correlation between zinc levels in muscle tissue, and % weight loss (R 0.72, p=0.001).

There was also a strong positive correlation between Zn levels in plasma and % weight loss (R 0.95, p=0.001). This is a very strong correlation.

It was shown that the plasma albumin concentration exhibited a negative significant relationship between % weight loss and plasma albumin levels (R −0.49.p=0.010).

There was also an association between the decrease in plasma albumin levels and the increase in plasma zinc levels (R −0.43, p=0.002).

Further, it was shown that there is a correlation between calprotectin fraction S 100 A9 and weight loss (R=0.61.p=0.016).

The S 100 A9 protein levels were also estimated in plasma samples using Western blotting. There was a positive correlation with % weight loss (R 0.7, p<0.001).

Skeletal muscle biopsies were also probed for S 100 A9 and there was a significant association with % weight loss (R 0.90, p=0.002).

As far as the present inventors are aware, these results are the first that are based on a systemic study measuring the concentrations of zinc and the calprotectin fraction S 100 A9 in the skeletal muscle tissue and plasma. The results show varying degrees of correlation with weight loss.

The decision to analyse these parameters which allow the surprising discovery underlying this description, is based on a finding made many years ago, namely that experimental animals of a certain age, in particular older mice, rapidly lost weight and exhibited leakage of endothelial tissue and dysfunction of the coagulation cascade and fibrinogen/fibrin metabolism. This finding motivated a deeper analysis of tyrosine phosphatase metabolism, an enzyme that can be inhibited by Zn²⁺.

Further, in Ebola virus infections, the tight junction structure seems to be affected and its function disturbed. It is known that epithelial cells typically form sheets, tubes, vesicles and other structures. These include apical and lateral cell surfaces, possessing several different functions.

The basolateral surface rests on a base membrane. Epithelial cells associate with each other through specialized molecules and structures, including the so called tight junction, adherence junctions and desmosomes.

Tight junctions can be described as membrane fusions at the lateral side, nearest to the apical surface that provides intercellular sealing and protection against cellular liquid diffusion, and leakage of molecules such as albumin. The tight junctions also help separating and distinguishing the different functions of the basolateral cell surfaces.

Dysfunction of these important structures has very dangerous consequences. One approach to counteract this, and to re-balance the status, is based on the balancing of MMP-9 enzyme activity and blocking the inhibitory effect of Zn ions against tyrosine phosphatase activity.

Compounds according to this description, such as alpha trinositol and derivatives thereof, when administered i.v. in animal experiments, such as BALB/c mice, have a counterbalancing effect. There is strong evidence that alpha trinositol and derivatives thereof have an effect on the tight junction and the capacity to counteract the disturbances.

Different processes, such as mesenchymal to epithelial transition, can be studied in in vitro and in vivo experiments, and the role of for example TGF beta. Also the function and behaviour of MET, under pathological conditions and in response to treatment, can be studied in in vitro and in vivo experiments. Compounds according to this description, such as alpha trinositol and derivatives thereof can be administered in non-toxic concentrations either i.v. or per OS.

### Materials

Alpha-trinositol (1-D-Myo-inositol 1,2,6-triphosphate) was prepared according to U.S. Pat. No. 4,777,134. In a glass ampoule a stock solution was prepared by dissolving 1 g of alpha-trinositol in saline to a total volume of 10 ml. The stock solution was stored in a refrigerator for use within 24 hrs.

### In Vivo Experiments

In planning animal experiments, one can start from the knowledge that infection with Ebola virus subtype Zaire produces fatal, severe illness in nonhuman primates, guinea pigs and young, suckling mice. However, guinea pigs inoculated with infectious virus material taken from patients infected with Ebola virus usually develop usually a nonlethal febrile illness.

It has been shown in laboratory experiments that the virulence increases after sequential passage of splenic tissue homogenates taken from infected animal to animal, resulting in high lethality. It has been shown that it is possible to increase the lethality nearly ten-fold in a time frame of 7 to 10 days. Thus, it is possible to regulate the lethality and develop a useful animal model, using guinea pigs.

It is also known from animal experiments, testing Ebola infection through different routes, that newborn mice are very sensitive to Ebola infection, and not only through intraperitoneal application. However, because newborn mice cannot be routinely used to study the pathogenesis of Ebola, involving different analytical procedures and the need of repeated taking of samples, there is a need to develop practical and useful animal models for the study of Ebola infection. One important consideration is that the animal model needs to be an animal having an adequate and functioning immune system, and this is not developed in suckling mice.

It seems to be a fact that that adult immunocompetent mice are nearly solidly resistant to wild-type filoviruses, regardless of the route of inoculation. This has however been resolved by adaptation of filoviruses for mice through sequential passage. Also, the best route of application has been found to be intraperitoneal (i.p.). There are many theories why it is so, but results clarifying the mode of action are not available yet.

Most importantly, the disease developed in this animal model, adult immunocompetent mice by i.p. inoculation of Ebola virus resembles Ebola infection in nonhuman primates. The virus replicates rapidly, with serum titers reaching at least 10 pfu/mL (pfu=plaque forming units)

Thus, serial passage of Ebola virus can be used to develop an experimental test in mice. Adult female mice BALB/c, C57BL/6 and ICR mice can be used in an experiment to test the effect of the compounds according to the current description, for example inositol triphosphate, against Ebola infection.

After serial passages of virus applied i.p. to the test animals, eight to nine (8-9) days later the animals were killed and the livers and spleens were aseptically removed and pooled. Tissue samples are then ground in a mortar with standardized sterile sand and growth medium, and the suspension is clarified by low-speed centrifugation in cold environment and titrated.

In passages two through eight, the pooled liver/spleen suspensions are inoculated i.p. in the experimental animals.

### Evaluation of Lethality in Mice

Lethality is determined by diluting Ebola virus in growth medium and inoculating i.p. In other experiments, groups of 16 days old mice are given serial 10-fold dilutions of virus in four different experiments analysing the combined results using linear regression software.

### Histology Studies

Portions of liver and spleen are fixed for 20-30 days in 10% neutral formalin buffer solution and then embedded in paraffin. Sections are mounted on glass slides and stained immunohistochemically.

Necropsy tissues from EBO-infected monkeys are used as positive controls. Liver and spleen samples from uninfected mice are used as negative controls. The results are tabulated and detailed.

The development of ruffled fur, slowed reactivity, symptoms of stiff neck, cramped posture and lack of motility is recognized in with comparison with control animals.

In summary, the Ebola virus infection mouse model is tested, and found to be functioning well. It has many desirable technical features for an animal model for investigating of an exceptionally virulent human disease.

The very fast progression of this disease often leads to a fatal end in only 5 to 8 days or even less. It is important that the test is conducted by experienced personnel, and that the virus is applied i.p. in a standardized fashion, ensuring that the animals develop the disease within three (3) days from infection (the i.p. application of virus).

When the animals used are genetically identical and of the same sex and same age, the results are highly reproducible and this makes the statistic evaluation of the effects and the therapeutic value very reliable.

One of the most important questions to be evaluated in an animal test is to study in detail the coagulation cascade. An enigmatic feature of the Ebola mouse test is the disparity between the application routes, i.e. intraperitoneal (i.p.) versus peripheral inoculation. This difference has not been observed in guinea pigs or

Non-human primates, who can be effectively inoculated s.c., i.m. or i.p. It seems to be so, that virus delivered into the peritoneal cavity are rapidly distributed through the peritoneal vessels—lymphatic vessels—and enter the blood circulation. This allows the virions to reach and infect the mononuclear phagocyte system (MPS) cells in the liver and spleen.

The mouse model may also prove to be very useful and valuable as an experimental research tool to study other pathogenic microorganisms exhibiting the epithelial tissue leakage phenomenon. Mice can be easily monitored in such experiments, after the application of an infectious agent i.p. and at the same time administering a drug candidate per os to the test animal.

### Animal Test Using BALC/Mice as Test Animals

6 weeks old BALC/mice are used to test the therapeutic effect of compounds according to the present description, e.g. alpha-trinositol, administered i.p. at very tolerable doses.

Groups of 10 mice are infected i.p. with 1,000 50% lethal doses (LD50 10 focus forming units (ffu) of mouse adapted Ebola virus by the intraperitoneal route).

The treatment involving the drug to be tested is delivered 1 h after virus administration i.p. The doses are calculated as mg/kg body weight. The drug is administered daily (i.p.) during six (6) days.

The mice are monitored for signs of disease during the test period according to the following criteria: ruffled fur, cramped posture, symptoms of diarrhea. The secretion of transforming growth factor Beta (TGF-b) is monitored before, during and after the experimental period. Similarly, the secretion of metalloproteinase 9 was observed, in order to analyze the transition phenomena of epithelium to mesenchyme transition.

The activity of tyrosine phosphatase enzyme is also analyzed and in that context, the concentration of Zn²⁺ in plasma samples is also determined.

A key to resolve the enigmatic mechanism of this fatal diseases is to analyze the dyshomeostasis of kinase-mediated phosphorylation events, and to investigate the effect of compounds according to this description, e.g. alpha-trinositol, and their effect to inhibit the Zn ions from blocking tyrosine phosphatase activity locally at, e.g. at the epithelial and endothelial blood vessels. Re-balancing the opening of the tight junction between endothelial cells which have been phosphorylated by tyrosine kinase enzyme and locally provoked via transforming factor beta 8 TG F-beta and by at the same cascade up-regulated matrix metalloproteinase-9 activity.

There is a difference between totally blocking the activity of tyrosine phosphatase (CD 45) and blocking its effect by Zn²⁺, and counterbalancing that effect by chelating Zn using the compounds described herein, e.g. alpha-inositol. It is contemplated by the present inventor that the total inhibition of tyrosine phosphatase activity is too manifest in the living body.

The current understanding of Ebola virus pathogenesis is that the virus, after entering the host animal, targets macrophages and dendritic cells thereby inducing an inflammation state with high levels of pro-inflammatory cytokines. At the same time the virus evades the immuneresponse by several mechanisms, including IFN antagonism, followed by depletion of NK cells and lymphocytes and defective DC function.

The following phase is believed to be the production of cytokines and triggering the expression of TF (tissue factor) thus provoking a pro-coagulant state, which then also has an enhancing effect on inflammation. The pro-coagulation state develops into DIC (disseminated intravenous coagulation> and further the released cytokines impair the endothelial barrier line and function. This usually leads to severe chock and death.

Genomic analysis has shown many similarities in infection induced alterations of mRNA expression including that of coagulation and acute phase related genes, as well as an overall antagonism of key responses, including IFN regulatory factor 3, PKR and TLR pathways.

It is of great significance that these viruses are able to control host cell mRNA expression and to prevent activation of the innate antiviral response.

The Ebola virus has been shown to have an ability to evade the cellular antiviral response and suppress the ability of this virus to alter the host cell gene expression program. As example of this is that one of the principal components of the innate immune and anti-viral responses is exactly this type of INF 1 response.

In this context it should be remembered that the 1918 pandemic influenza virus revealed a higher INF antagonistic activity of the pandemic NSI protein compared to other HINI viruses (Geiss G. K. et al. Proc. Natl. Acad. Sci. USA, 99, 10736-10741).

Although particular embodiments have been disclosed herein in detail, this has been done by way of example for purposes of illustration only, and is not intended to be limiting with respect to the scope of the appended claims that follow. In particular, it is contemplated by the inventor that various substitutions, alterations, and modifications may be made without departing from the spirit and scope of the invention as defined by the claims.

### Listing of Aspects and Embodiments of the Invention

1. Use of a compound comprising a high density, negatively charged domain of vicinally oriented radicals for preventing, alleviating and/or treating filovirus infection in a mammal including man.
2. The use according to claim 1, wherein the filovirus infection is an infection caused by a virus chosen from Marburg virus, Cueva virus, and Ebola virus families.
3. The use according to claim 1, wherein the filovirus infection is an infection caused by an Ebola virus.
4. The use according to claim 1, wherein the negatively charged domain comprises three or more vicinal phosphorus-containing radicals.
5. The use according to claim 1, wherein the phosphorus-containing radical has the general formula I

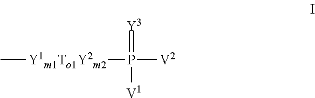

or the general formula II

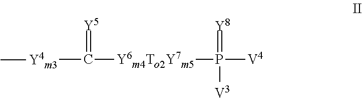

where

### V¹ to V⁴ are Y⁹ₘ₆Tₒ₃U

Tₒ₁ to Tₒ₃ are (CH₂)ₙ, CH═CH, or CH₂CH═CHCH₂
o1 to o3 are 0 to 1
n is 0 to 4
U is R¹Y¹⁰ₘ₇, CY¹¹Y¹²R², SY¹³Y¹⁴Y¹⁵R³, PY¹⁶Y¹⁷Y¹⁸R⁴R⁵, Y¹⁹PY²⁰Y²¹Y²²R⁶R⁷, CH₂NO₂, NHSO₂R⁸ or NHCY²³Y²⁴R⁹
m1 to m7 are 0 to 1

### Y¹ to Y²⁴ are NHR¹⁰, NOR¹¹, O or S

and where R¹ to R¹¹ are
i. hydrogen;
ii. a straight or branched saturated or unsaturated alkyl residue of 1-22 carbon atoms;
iii. a saturated or unsaturated aromatic or non-aromatic homo- or heterocyclic residue of 3-22 carbon atoms and 0-5 hetero atoms selected from nitrogen, oxygen and sulphur;
iv. a straight or branched saturated or unsaturated alkyl residue of 1-22 carbon atoms comprising a saturated or unsaturated aromatic or non-aromatic homo- or heterocyclic substituent of 3-22 carbon atoms and 0-5 hetero atoms selected from nitrogen, oxygen and sulphur;
v. an aromatic or non-aromatic homo- or heterocyclic residue of 3-22 carbon atoms and 0-5 heteroatoms selected from nitrogen, oxygen and sulphur, comprising a straight or branched saturated or unsaturated alkyl substituent of 1-22 carbon atoms.
6. The use according to claim 5, wherein one or several of the residues and/or substituents of R1 to R11, ii)-v), are substituted with 1 to 6 of hydroxy, alkoxy, aryloxy, acyloxy, carboxy, alkoxycarbonyl, alkoxycarbonyloxy, aryloxycarbonyl, aryloxycarbonyloxy, carbamoyl, fluoro, chloro, bromo, azido, cyano, oxo, oxa, amino, imino, alkylamino, arylamino, acylamino, arylazo, nitro, alkylthio and alkylsulfonyl.
7. The use according to claim 5, wherein one or several of the straight or branched saturated or unsaturated alkyl residues and substituents of R1 to R11, ii), iv), v) are selected from the group consisting of methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, heneicosyl, doeicosyl, isopropyl, isobutyl, isopentyl, isohexyl, isoheptyl, isooctyl, isononyl, isodecyl, isodoecosyl, 2-butyl, 2-pentyl, 2-hexyl, 2-heptyl, 2-octyl, 2-nonyl, 2-decyl, 2-doeicosyl, 2-methylbutyl, 2-methylpentyl, 2-methylhexyl, 2-methyl-heptyl, 2-methyloctyl, 2-methylnonyl, 2-methyldecyl, 2-methyl-eicosyl, 2-ethylbutyl, 2-ethylpentyl, 2-ethyl-hexyl, 2-ethylheptyl, 2-ethyloctyl, 2-ethylnonyl, 2-ethyldecyl, 2-ethyleicosyl, tertbutyl, ethenyl, propenyl, butenyl, pentenyl, hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, heneicosenyl, doeicosenyl, butadienyl, pentadienyl, hexadienyl, heptadienyl, octadienyl, nonadienyl, decadienyl, doeicodienyl, ethynyl, propynyl, and doeicosynyl.
8. The use according to claim 5, wherein a saturated aromatic or non-aromatic homo- or heterocyclic of R¹ to R¹¹, iii)-v), is selected from the group consisting of cyclopropyl, cyclobutyl, cyclopentyl, cyclohexyl, cycloheptyl, cyclooctyl, cyclononyl, cyclodecyl, cycloundecyl, cyclododecyl, cycloridecyl, cyclotetradecyl, cyclopentadecyl, cyclohexadecyl, cycloheptadecyl, cyclooctadecyl, cyclononadecyl, cycloeicosyl, cycloheneicosyl, cyclodoeicosyl, adamantyl, cyclopropenyl, cyclobutenyl, cyclopentenyl, cyclohexenyl, cycloheptenyl, cyclooctenyl, cyclononenyl, cyclodecenyl, phenyl, biphenyl, naphthyl, hydroxyphenyl, aminophenyl, mercaptophenyl, fluorophenyl, chlorophenyl, azidophenyl, cyanophenyl, carboxyphenyl, alkoxyphenyl, acyloxyphenyl, acylphenyl, oxiranyl, thiiranyl, aziridinyl, oxetanyl, thietanyl, azetidinyl, tetrahydrofuranyl, tetrahydrothiophenyl, pyrrolidinyl, tetrahydropyranyl, tetrahydrothiopyranyl, piperidinyl, quinuclidinyl, dioxanyl, dithianyl, trioxanyl, furyl, pyrrolyl, thienyl, pyridyl, quinolyl, benzofuryl, indolyl, benzothienyl, oxazolyl, imidazolyl, thiazolyl, pyridazinyl, pyrimidyl, pyrazinyl, purinyl and a carbohydrate.
9. The use according to claim 4, wherein a phosphorus-containing radical of the invention has the general formula III

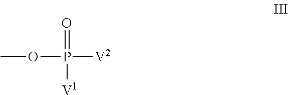

where V¹ and V² are, independent of each other, selected from the group consisting of OH, (CH₂)ₚOH, COOH, CONH₂, CONOH, (CH₂)ₚCOOH, (CH₂)ₚCONH₂, (CH₂)ₚCONOH, (CH₂)ₚSO₃H, (CH₂)ₚSO₃, NH₂, (CH₂)ₚNO₂, (CH₂)ₚPO₃H₂, O(CH₂)ₚOH, O(CH₂)ₚCOOH, O(CH₂)ₚCONH₂, O(CH₂)ₚCONOH, (CH₂)ₚSO₃H, O(CH₂)ₚSO₃NH₂, O(CH₂)ₚNO₂, O(CH₂)ₚPO₃H₂, and CF₂COOH and p is 1 to 4.
10. The use according to any one of claim 1,4 or 9, wherein the compound of the invention is a pharmaceutically acceptable phosphate, phosphonate or phosphinate.
11. The use according to claim 10, wherein the compound is a sodium, potassium, calcium or magnesium salt of the phosphate, phosphonate or phosphinate.
12. The use according to any one of claims 9-11, wherein the domain of high density negatively charged vicinally oriented radicals is linked to a cyclic moiety comprising or consisting of a saturated or unsaturated aromatic or non-aromatic homo- or heterocyclic ring.
13. The use according to claim 12, wherein heteroatom(s) of the heterocyclic ring of the cyclic moiety is/are, independently of each other, selected from the group consisting of oxygen, nitrogen, sulphur, and selenium.
14. The use according to claim 12, wherein the cyclic moiety comprises from 4 to 24 atoms, in particular from 5 to 18 atoms, and most particularly 6 atoms.
15. The use according to any one of claims 12-14, wherein the cyclic moiety is selected from the group consisting of cyclopentane, cyclohexane, cycloheptane, cyclooctane, inositol, monosaccharide, disaccharide, trisaccharide, tetrasaccharide, arabinitol, piperidine, tetra-hydrothiopyran, 5-oxotetrahydrothiopyran, 5,5-dioxotetrahydro-thiopyran, tetrahydroselenopyran, tetrahydrofuran, pyrrolidine, tetrahydrothiophene, 5-oxotetrahydrothiophene, 5,5-dioxotetrahydrothiophene, tetrahydroselenophene, benzene, cumene, mesitylene, naphtalene and phenantrene.
16. The use according to claim 15, wherein the cyclic moiety comprises one or more hydroxyl groups not bound to phosphorous-containing radicals of which at least one is derivatized in form of ether or ester.
17. The use according to any one of claim 15 or 16, wherein the cyclic moiety is selected from the group consisting of inositol, monosaccharide, disaccharide, trisaccharide, and tetrasaccharide.
18. The use according to claim 17, wherein the cyclic moiety is an inositol selected from the group consisting of allo-inositol, cis-inositol, epi-inositol, D/L-chiro-inositol, scylloinositol, myoinositol, mucoinositol and neoinositol.
19. The use according to any one of claims 17-18, wherein the cyclic moiety is inositol and the compound is a phosphate, a phosphonate or a phosphinate of inositol.
20. The use according to claims 18-19, wherein the number of phosphate, phosphonate or phosphinate radicals per inositol moiety is three or more.
21. The use according to claim 17, wherein the cyclic moiety is inositol and the inositol is selected from the group consisting of inositol-trisphosphate, inositol-tris(carboxymetyl-phosphate), inositol-tris(carbomethylphosphonate), inositol-tris(hydroxymethylphosphonate), tri-O-methyl-inositol-trisphosphate, tri-O-hexyl-inositol-trisphosphate, tri-O-butyl-inositol-trisphosphate, tri-O-pentyl-inositol-trisphosphate, tri-O-isobutyl-inositol-trisphosphate, tri-O-propyl-inositol-trisphosphate, tri-O-(6-hydroxy-4-oxa)hexyl-inositol-trisphosphate, tri-O-3-(ethylsulfonyl)propyl-inositol-trisphosphate, tri-O-3-hydroxypropyl-inositol-trisphosphate, tri-O-(6-hydroxy)-hexyl-inositol-trisphosphate, tri-O-phenylcarbamoyl-inositol-trisphosphate, tri-O-propyl-inositol-tris(carboxymethylphosphate), tri-O-butyl-inositol-tris(carboxymethylphosphate), tri-O-isobutyl-inositol-tris(carboxymethyl-phosphate), tri-O-pentyl-inositol-tris(carboxymethylphosphate), tri-O-hexyl-inositol-tris(carboxymethylphosphate), tri-O-propyl-inositol-tris(carboxymethylphosphonate), tri-O-butyl-inositol-tris(carboxymethyl-phosphonate), tri-O-isobutyl-inositol-tris(carboxymethylphosphonate), tri-O-pentyl-inositol-tris(carboxymethylphosphonate), tri-O-hexyl-inositol-tris(carboxymethylphosphonate), tri-O-propyl-inositol-tris(hydroxymethyl-phosphonate), tri-O-butyl-inositol-tris(hydroxymethylphosphonate), tri-O-isobutyl-inositol-tris(hydroxymethylphosphonate), tri-O-pentyl-inositol-tris(hydroxymethylphosphonate), and tri-O-hexyl-myo-inositol-tris(hydroxymethyl-phosphonate).
22. The use according to claim 21, wherein the compound is inositol triphosphate.
23. The use according to claim 18, wherein the cyclic moiety is myoinositol and said myoinositol is selected from the group consisting of D-myo-inositol-1,2,6-trisphosphate, D-myo-inositol-1,2,6-tris(carboxymetyl-phosphate), D-myo-inositol-1,2,6-tris(carbomethylphosphonate), D-myo-inositol-1,2,6-tris(hydroxymethylphosphonate), D-3,4,5-tri-O-methyl-myo-inositol-1,2,6-trisphosphate, D-3,4,5-tri-O-hexyl-myo-inositol-1,2,6-trisphosphate, D-3,4,5-tri-O-butyl-myo-inositol-1,2,6-trisphosphate, D-3,4,5-tri-O-pentyl-myo-inositol-1,2,6-trisphosphate, D-3,4,5-tri-O-isobutyl-myo-inositol-1,2,6-trisphosphate, D-3,4,5-tri-O-propyl-myo-inositol-1,2,6-trisphosphate, D-3,4,5-tri-O-(6-hydroxy-4-oxa)hexyl-myo-inositol-1,2,6-trisphosphate, D-3,4,5-tri-O-3-(ethylsulfonyl)propyl-myo-inositol-1,2,6-trisphosphate, D-3,4,5-tri-O-3-hydroxypropyl-myo-inositol-1,2,6-trisphosphate, D-3,4,5-tri-O-(6-hydroxy)-hexyl-myo-inositol-1,2,6-trisphosphate, D-5-O-hexyl-myo-inositol-1,2,6-trisphosphate, D-3,4,5-tri-O-phenylcarbamoyl-myo-inositol-1,2,6-trisphosphate, D-3,4,5-tri-O-propyl-myo-inositol-1,2,6-tris(carboxymethylphosphate), D-3,4,5-tri-O-butyl-myo-inositol-1,2,6-tris(carboxymethylphosphate), D-3,4,5-tri-O-isobutyl-myo-inositol-1,2,6-tris(carboxymethyl-phosphate), D-3,4,5-tri-O-pentyl-myo-inositol-1,2,6-tris(carboxymethylphosphate), D-3,4,5-tri-O-hexyl-myo-inositol-1,2,6-tris(carboxymethylphosphate), D-3,4,5-tri-O-propyl-myo-inositol-1,2,6-tris(carboxymethylphosphonate), D-3,4,5-tri-O-butyl-myo-inositol-1,2,6-tris(carboxymethyl-phosphonate), D-3,4,5-tri-O-isobutyl-myo-inositol-1,2,6-tris(carboxymethylphosphonate), D-3,4,5-tri-O-pentyl-myo-inositol-1,2,6-tris(carboxymethylphosphonate), D-3,4,5-tri-O-hexyl-myo-inositol-1,2,6-tris(carboxymethylphosphonate), D-3,4,5-tri-O-propyl-myo-inositol-1,2,6-tris(hydroxymethyl-phosphonate), D-3,4,5-tri-O-butyl-myo-inositol-1,2,6-tris(hydroxymethylphosphonate), D-3,4,5-tri-O-isobutyl-myo-inositol-1,2,6-tris(hydroxymethylphosphonate), D,-3,4,5-tri-O-pentyl-myo-inositol-1,2,6-tris(hydroxymethylphosphonate), D-3,4,5-tri-O-hexyl-myo-inositol-1,2,6-tris(hydroxymethyl-phosphonate), D-3,4,5-tri-O-propanoyl-myo-inositol-1,2,6-tris(carboxymethylphosphate), D-3,4,5-tri-O-butanoyl-myo-inositol-1,2,6-tris(carboxymethylphosphate), D-3,4,5-tri-O-isobutanoyl-myo-inositol-1,2,6-tris(carboxymethyl-phosphate), D-3,4,5-tri-O-pentanoyl-myo-inositol-1,2,6-tris(carboxymethylphosphate), D-3,4,5-tri-O-hexanoyl-myo-inositol-1,2,6-tris(carboxymethylphosphate), D-3,4,5-tri-O-propanoyl-myo-inositol-1,2,6-tris(carboxymethylphosphonate), D-3,4,5-tri-O-butanoyl-myo-inositol-1,2,6-tris(carboxymethyl-phosphonate), D-3,4,5-tri-O-isobutanoyl-myo-inositol-1,2,6-tris(carboxymethylphosphonate), D-3,4,5-tri-O-pentanoyl-myo-inositol-1,2,6-tris(carboxymethylphosphonate), D-3,4,5-tri-O-hexanoyl-myo-inositol-1,2,6-tris(carboxymethylphosphonate), D-3,4,5-tri-O-propanoyl-myo-inositol-1,2,6-tris(hydroxymethyl-phosphonate), D-3,4,5-tri-O-butanoyl-myo-inositol-1,2,6-tris(hydroxymethylphosphonate), D-3,4,5-tri-O-isobutanoyl-myo-inositol-1,2,6-tris(hydroxymethylphosphonate), D,-3,4,5-tri-O-pentanoyl-myo-inositol-1,2,6-tris(hydroxymethylphosphonate), and D-3,4,5-tri-O-hexanoyl-myo-inositol-1,2,6-tris(hydroxymethyl-phosphonate).
24. The use according to claim 23, wherein the cyclic moiety is inositol, said inositol being myoinsoitol and said myoinositol is myo-inositol-1,2,6-trisphosphate or myo-inositol-1,2,3-trisphosphate.
25. The use according to claim 24, wherein the compound is the pentasodium salt of 1,2,6-D-myo inositol trisphosphate (Na₅H 1,2,6-D-myo-inositol trisphosphate), Mg₃ 1,2,6-D-myo-inositol trisphosphate or Ca₃ 1,2,6-D-myo-inositol trisphosphate).
26. The use according to any one of claims 16-17, wherein at least one of the hydroxyl groups is derivatized to form an ester having the general formula IV

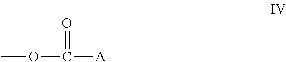

27. The use according to claim 26, wherein A is a straight or branched saturated or unsaturated alkyl residue containing 1 to 24 carbon atoms to be selected from the group consisting of methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, heneicosyl, doeicosyl, isopropyl, isobutyl, isopentyl, isohexyl, isoheptyl, isooctyl, isononyl, isodecyl, isodoecosyl, 2-butyl, 2-pentyl, 2-hexyl, 2-heptyl, 2-octyl, 2-nonyl, 2-decyl, 2-doeicosyl, 2-methylbutyl, 2-methylpentyl, 2-methylhexyl, 2-methylheptyl, 2-methyloctyl, 2-methylnonyl, 2-methyldecyl, 2-methyleicosyl, 2-ethylbutyl, 2-ethylpentyl, 2-ethylhexyl, 2-ethylheptyl, 2-ethyloctyl, 2-ethylnonyl, 2-ethyldecyl, 2-ethyleicosyl, tert-butyl, ethenyl, propenyl, butenyl, pentenyl, hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, heneicosenyl, doeicosenyl, butadienyl, pentadienyl, hexadienyl, heptadienyl, octadienyl, nonadienyl, decadienyl, doeicodienyl, ethynyl, propynyl and doeicosynyl.
28. The use according to claim 26, wherein A is a saturated or unsaturated aromatic or non-aromatic homo- or heterocyclic residue or substituent to be selected from the group consisting of cyclopropyl, cyclobutyl, cyclopentyl, cyclohexyl, cycloheptyl, cyclooctyl, cyclononyl, cyclodecyl, cycloundecyl, cyclododecyl, cycloridecyl, cyclotetradecyl, cyclopentadecyl, cyclohexadecyl, cycloheptadecyl, cyclooctadecyl, cyclononadecyl, cycloeicosyl, cycloheneicosyl, cyclodoeicosyl, adamantyl, cyclopropenyl, cyclobutenyl, cyclopentenyl, cyclohexenyl, cycloheptenyl, cyclooctenyl, cyclononenyl, cyclodecenyl, phenyl, biphenyl, naphthyl, hydroxyphenyl, aminophenyl, mercaptophenyl, fluorophenyl, chlorophenyl, azidophenyl, cyanophenyl, carboxyphenyl, alkoxyphenyl, acyloxyphenyl, acylphenyl, oxiranyl, thiiranyl, aziridinyl, oxetanyl, thietanyl, azetidinyl, tetrahydrofuranyl, tetrahydrothiophenyl, pyrrolidinyl, tetrahydropyranyl, tetrahydrothiopyranyl, piperidinyl, quinuclidinyl, dioxanyl, dithianyl, trioxanyl, furyl, pyrrolyl, thienyl, pyridyl, quinolyl, benzofuryl, indolyl, benzothienyl, oxazolyl, imidazolyl, thiazolyl, pyridazinyl, pyrimidyl, pyrazinyl, purinyl and carbohydrate.
29. The use according to claim 26 wherein A is (CH₂)ₙOR¹² where n is an integer between 1 and 10 and where R¹² is hydrogen, a substituted or unsubstituted straight or branched alkyl, cycloalkyl, aryl or alkaryl; preferably n is between 2 and 4 and R¹² is hydrogen or a lower alkyl such as methyl, ethyl or propyl.
30. The use according to claim 26, wherein A is (CH₂)ₙZ(CH₂)ₘOR¹² where n and m is an integer between 1 and 10, where Z is oxygen or sulphur and where R¹² is hydrogen, a substituted or unsubstituted straight or branched alkyl, cycloalkyl, aryl or alkaryl; preferably n is 1, m is between 2 and 4 and R¹² is hydrogen or a lower alkyl such as methyl, ethyl or propyl.
31. The use according to claim 26, wherein A is (CH₂)ₙOCOR¹² where n is an integer between 1 and 10 and where R¹² is hydrogen, a substituted or unsubstituted straight or branched alkyl, cycloalkyl, aryl or alkaryl; preferably n is between 2 and 4 and R¹² is hydrogen or a lower alkyl such as methyl, ethyl or propyl.
32. The use according to claim 26, wherein A is (CH₂)ₙCOOR¹² where n is an integer between 1 and 10 and where R¹² is hydrogen, a substituted or unsubstituted straight or branched alkyl, cycloalkyl, aryl or alkaryl; preferably n is between 2 and 4 and R¹² is hydrogen or a lower alkyl such as methyl, ethyl or propyl.
33. The use according to claim 26, wherein A is (CH₂)ₙOCOOR¹² where n is an integer between 1 and 10 and where R¹² is hydrogen, a substituted or unsubstituted straight or branched alkyl, cycloalkyl, aryl or alkaryl; preferably n is between 2 and 4 and R¹² is hydrogen or a lower alkyl such as methyl, ethyl or propyl.
34. The use according to claim 26, wherein A is (CH₂)ₙR¹² where n is an integer between 1 and 10 and where R¹² is hydrogen, a substituted or unsubstituted straight or branched alkyl, cycloalkyl, aryl or alkaryl; preferably n is between 2 and 4 and R¹² is hydrogen, a lower alkyl such as methyl, ethyl or propyl.
35. The use according to claim 26, wherein A is (CH₂)ₙ OCONR¹²R¹³ where n is an integer between 1 and 10 and where R¹² and R¹³ are hydrogen, a substituted or unsubstituted straight or branched alkyl, cycloalkyl, aryl or alkaryl; preferably n is between 2 and 4 and R¹² and R¹³ are hydrogen or a lower alkyl such as methyl, ethyl or propyl.
36. The use according to any one of claims 15,16 and 26-35, wherein the compound is a phosphate, a phosphonate or a phosphinate of cyclohexane
37. The use according to claim 36, wherein the compound is 1,2,3-β-cyclohexane-1,2,3-trioltrisphosphate.
38. The use according to any one of claims 15-17 and 26, wherein the inositol triphosphate is selected from the group consisting of tri-O-hexanoyl-inositol-trisphosphate, tri-O-butanoyl-inositol-trisphosphate, tri-O-pentanoyl-inositol-trisphosphate, tri-O-(4-hydroxy)pentanoyl-inositol-trisphosphate, tri-O-isobutanoyl-inositol-trisphosphate, tri-0-propanoyl-inositol-trisphosphate, tri-O-(6-hydroxy-4-oxa)hexanoyl-inositol-trisphosphate, tri-O-3-(ethylsulfonyl)propanoyl-inositol-trisphosphate, tri-O-3-hydroxypropanoyl-inositol-trisphosphate, tri-O-(6-hydroxy)-hexanoyl-inositol-trisphosphate, tri-O-phenylcarbamoyl-inositol-trisphosphate, tri-O-dodecanoyl-inositol-trisphosphate, tri-O-(2-acetoxy) benzoylcarbamoyl-inositol-trisphosphate, tri-O-butylcarbamoyl-inositol-trisphosphate, tri-O-metylcarbamoyl-inositol-trisphosphate, and tri-O-phenylcarbamoyl-inositol-trisphosphate.
39. The use according to any one of claims 15-17 and 26, wherein the compound is selected from the group consisting of D-3,4,5-tri-O-hexanoyl-myo-inositol-1,2,6-trisphosphate, D-3,4,5-tri-O-butanoyl-myo-inositol-1,2,6-trisphosphate, D-3,4,5-tri-O-pentanoyl-myo-inositol-1,2,6-trisphosphate, D-3,4,5-tri-O-(4-hydroxy)pentanoyl-myo-inositol-1,2,6-trisphosphate, D-3,4,5-tri-O-isobutanoyl-myo-inositol-1,2,6-trisphosphate, D-3,4,5-tri-O-propanoyl-myo-inositol-1,2,6-trisphosphate, D-3,4,5-tri-O-(6-hydroxy-4-oxa)hexanoyl-myo-inositol-1,2,6-trisphosphate, D-3,4,5-tri-O-3-(ethylsulfonyl)propanoyl-myo-inositol-1,2,6-trisphosphate, D-3,4,5-tri-O-3-hydroxypropanoyl-myo-inositol-1,2,6-trisphosphate, D-3,4,5-tri-O-(6-hydroxy)-hexanoyl-myo-inositol-1,2,6-trisphosphate, D-3,4,5-tri-O-phenylcarbamoyl-myo-inositol-1,2,6-trisphosphate, D-3,4,5-tri-O-dodecanoyl-myo-inositol-1,2,6-trisphosphate, D-3,4,5-tri-O-(2-acetoxy) benzoylcarbamoyl-myo-inositol-1,2,6-trisphosphate, D-3,4,5-tri-O-butylcarbamoyl-myo-inositol-1,2,6-trisphosphate, D-3,4,5-tri-O-metylcarbamoyl-myo-inositol-1,2,6-trisphosphate, and D-3,4,5-tri-O-phenylcarbamoyl-myo-inositol-1,2,6-trisphosphate.
40. The use according to any one of claims 38-39, wherein the compound is in the form of its sodium salt.
41. The use according to claim 40, wherein the compound of the invention is penta sodium D-3,4,5-tri-O-hexanoyl-myo-inositol-1-2, 6-trisphosphate.
42. The use according to any one of claims 17 and 26-35, wherein the cyclic moiety is a mono- or disaccharide selected from the group consisting of D/L-ribose, D/L-arabinose, D/L-xylose, D/L-lyxose, D/L-allose, D/L-altrose, D/L-glucose, D/L-mannose, D/L-gulose, D/L-idose, D/L-galactose, D/L-talose, D/L-ribulose, D/L-xylulose, D/L-psicose, D/L-sorbose, D/L-tagatose and D/L-fructose or a derivative thereof.
43. The use according to claim 42, wherein the compound is a phosphate, a phosphonate or a phosphinate of a mono- or disaccharide.
44. The use according to claim 43, wherein the number of phosphate, phosphonate or phosphinate radicals per saccharide moiety is three or more.
45. The use according to any one of claims 42-44, wherein the compound comprising a saccharide moiety is selected from the group consisting of mannose-2,3,4-trisphosphate, galactose-2,3,4-trisphosphate, fructose-2,3,4-trisphosphate, altrose-2,3,4-trisphosphate and rhamnose-2,3,4-trisphosphate.
46. The use according to claims 15-17, wherein the compound is selected from the group consisting of R¹-6-O—R²-α-D-manno-pyranoside-2,3,4-trisphosphate, R¹-6-O—R²-α-D-galacto-pyranoside-2,3,4-trisphosphate, R¹-6-O—R²-α-D-altropyranoside-2,3,4-trisphosphate and R¹-6-O—R²-β-D-fructopyranoside-2,3,4-trisphosphate, wherein R¹ and R² independent of each other are selected from the group consisting of methyl, ethyl, propyl, butyl, pentyl or hexyl.
47. The use according to claim 46, wherein the compound is selected from the group consisting of methyl-6-O-butyl-α-D-mannopyranoside-2,3,4-trisphosphate, methyl-6-O-butyl-α-D-galactopyranoside-2,3,4-trisphosphate, methyl-6-O-butyl-α-D-glycopyranoside-2,3,4-trisphosphate, methyl-6-O-butyl-α-D-altropyranoside-2,3,4-trisphosphate, methyl-6-O-butyl-β-D-fructopyranoside-2,3,4-trisphosphate, 1,5-anhydro-D-arabinitol-2,3,4-trisphosphate, 1,5-anhydroxylitol-2,3,4-trisphosphate, 1,2-O-ethylene-β-D-fructopyranoside-2,3,4-trisphosphate, methyl-α-D-rhamno-pyranoside-2,3,4-trisphosphate, methyl-α-D-mannopyranoside-2,3,4-trisphosphate, methyl-6-O-butyl-α-D-mannopyranoside-2,3,4-tris-(carboxymethylphosphate), methyl-6-O-butyl-α-D mannopyranoside-2,3,4-tris(carboxymethylphosphonate), methyl-6-O-butyl-α-D-mannopyranoside-2,3,4-tris(hydroxymethyl-phosphonate), methyl-6-O-butyl-α-D-galactopyranoside-2,3,4-tris(carboxymethylphosphate), methyl-6-O-butyl-α-D-galacto-pyranoside-2,3,4-tris(carboxymethylphosphonate), methyl-6-O-butyl-α-D-galactopyranoside-2,3,4-tris(hydroxymethyl-phosphonate), methyl-6-O-butyl-α-D-glucopyranoside-2,3,4-tris(carboxymethylphosphate), methyl-6-O-butyl-α-D-glucopyranoside-2,3,4-tris(carboxymethylphosphonate), methyl-6-O-butyl-α-D-glucopyranoside-2,3,4-tris(hydroxy-methylphosphonate), methyl-6-O-butyl-α-D-altropyranoside-2,3,4-tris-(carboxymethylphosphate), methyl-6-O-butyl-α-D-altropyranoside-2,3,4-tris-(carboxymethylphosphonate), methyl-6-O-butyl-α-D-altropyranoside-2,3,4-tris-(hydroxyl-methylphosphonate), methyl-6-O-butyl-β-D-fructopyranoside-2,3,4-tris-(carboxymethylphosphate), methyl-6-O-butyl-β-D-fructopyranoside-2,3,4-tris-(carboxymethylphosphonate) and methyl-6-O-butyl-β-D-fructo-pyranoside-2,3,4-tris-(hydroxymethylphosphonate).
48. The use according to any one of claims 15 and 16, wherein the compound is a phosphate, phosphonate or phosphinate comprising a heterocyclic moiety that is an arabinitol and the arabinitols are selected from the group consisting of 1,5-dideoxy-1,5-iminoarabinitol-2,3,4-trisphosphate,1,5-dideoxy-1,5-iminoarabinitol-2,3,4-tris-(carboxymethylphosphate), 1,5-dideoxy-1,5-imino-arabinitol-2,3,4-tris(carboxymethyl-phosphonate), 1,5-dideoxy-1,5-iminoarabinitol-2,3,4-tris(hydroxymethylphosphonate), 1,5-dideoxy-1,5-imino-N-(2-phenylethyl)arabinitol-2,3,4-trisphosphate, 1,5-dideoxy-1,5-imino-N-(2-phenylethyl)-arabinitol-2,3,4-tris(carboxy-methylphosphate), 1,5-dideoxy-1,5-imino-N-(2-phenylethyl)-arabinitol-2,3,4-tris-(carboxy-methylphosphonate), and 1,5-dideoxy-1,5-imino-N-(2-phenyl-ethyl)arabinitol-2,3,4-tris(hydroxymethylphosphonate).
49. A pharmaceutical composition comprising the penta sodium salt of 1,2,6-D-myo inositol trisphosphate in a dose that is pharmacologically efficient in the treatment, alleviation or prevention of a disease caused by a virus of the family Filoviridae, in particular a dose of from about 1 mg/kg body weight to about 100 mg/kg body weight, preferably from about 10 mg to 60 mg/kg body weight, or a dose of corresponding efficiency of another compound used according to claims 1 to 48 and a pharmaceutically acceptable fluid carrier.
50. The composition according to claim 49, wherein the carrier is an aqueous carrier, in particular saline.
51. A pharmaceutical composition according to claim 49 or 50, wherein said virus is chosen from haemorrhagic viruses, such as the Ebola and Marburg viruses.
52. A pharmaceutical composition comprising a dose of 1,2,6-D-myo inositol trisphosphate that is pharmacologically efficient in the treatment, alleviation or prevention of a disease caused by a virus of the family Filoviridae, in particular a dose of from 5 mg to 80 mg of the pentasodium salt of 1,2,6-D-myo inositol trisphosphate, or a dose of corresponding efficiency of another compound used according to claims 1 to 48, in a closed container and in crystalline or amorphous form, including in form of a cryoprecipitate.
53. A composition according to claim 52, wherein said dose is dispersed in a stabilizing agent or a mixture of stabilizing agents, in particular in one or more of glucose, mannose, sodium chloride.
54. A pharmaceutical composition comprising a compound used according to claims 1 to 46, an analgesic agent and a pharmaceutically acceptable carrier.
55. A composition according to claim 54, wherein the analgesic agent is an opioid agonist.
56. A composition according to claim 54, wherein the opioid agonist is selected from morphine, nalorphine, nalbuphine; levorphanol, racemorphan, levallorphan, dextromethorphan, cyclorphan, butorphanol, pentazocine, phenazocine, cyclazocine, ketazocine, pethidine, meperidine diphenoxylate, anileridine, piminodine, fentanil, ethoheptazine, alphaprodine, betaprodine, 1-methyl-4-phenyl-1,2,5,6-tetrahydropyridine (MPTP), loperamide, sulfentanil, alfentanil, remifentanil, lofentanil, methadone, d-propoxyphene, isomethandone, levo-alpha-acetylmethadol (LAAM), naloxone, naltrexone, natrindole; oripavine and its derivatives, codeine, heterocodeine, morphinone, dihydromorphine, dihydrocodeine, dihydromorphinone, dihydrocodeinone, 6-desoxumorphine, oxymorphone, oxycodone, 6-methylene-dihydromorphine, hydrocodone, hydromorphone, metopon, apomorphine, normorphine, N-(2-phenylethyl)-normorphine, etorphine, buprenorphine, spiradoline, enadoline or asimadoline.
57. A composition according to claims 49-56, wherein the carrier is an aqueous carrier.
58. A nutritional composition for treating or alleviating the symptoms of a filovirus infection, or for preventing catabolic conditions associated with filovirus infection, comprising:
an inositol triphosphate or an ester thereof, or a mono- or disaccharide having three or more phosphate radicals per saccharide moiety or an ester thereof; and
at least one nutrient selected from the group consisting of lipid emulsions, fluid sources of amino acids and carbohydrates.
59. A nutritional composition according claim 58 adapted for parenteral administration.
60. A nutritional composition according to claim 58 adapted for oral or enteral administration.
61. A nutritional composition according to claim 58, wherein normal intake of the nutritional supplement provides 5 to 80 mg per kg body weight of inositol triphosphate or an ester thereof, or a mono- or disaccharide having three or phosphate radicals per saccharide moiety or an ester thereof are to a patient.

1. A method for preventing, alleviating and/or treating filovirus infection in a mammal including man in need of such treatment, which comprises administering an effective amount of a compound comprising a high density, negatively charged domain of vicinally oriented radicals.

2. The method according to claim 1, wherein the filovirus infection is an infection caused by a virus chosen from Marburg virus, Cueva virus, and Ebola virus families.

3. The method according to claim 1, wherein the filovirus infection is an infection caused by an Ebola virus.

4. The method according to claim 1, wherein the negatively charged domain comprises three or more vicinal phosphorus-containing radicals.

5. The method according to claim 1, wherein the phosphorus-containing radical has the general formula I 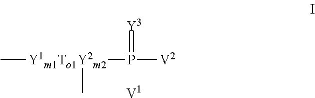
or the general formula II 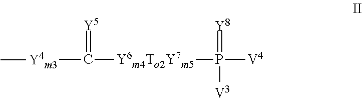
wherein
V¹ to V⁴ are Y⁹ₘ₆Tₒ₃U
Tₒ₁ to Tₒ₃ are (CH₂)ₙ, CH═CH, or CH₂CH═CHCH₂
o1 to o3 are 0 to 1
n is 0 to 4
U is R¹Y¹⁰ₘ₇, CY¹¹Y¹²R², SY¹³Y¹⁴Y¹⁵R³, PY¹⁶Y¹⁷Y¹⁸R⁴R⁵, Y¹⁹PY²⁰Y²¹Y²²R⁶R⁷, CH₂NO₂, NHSO₂R⁸ or NHCY²³Y⁴R⁹
m1 to m7 are 0 to 1
Y¹ to Y²⁴ are NHR¹⁰, NOR¹¹, O or S
and wherein R¹ to R¹¹ are
i. hydrogen;
ii. a straight or branched saturated or unsaturated alkyl residue of 1-22 carbon atoms;
iii. a saturated or unsaturated aromatic or non-aromatic homo- or heterocyclic residue of 3-22 carbon atoms and 0-5 hetero atoms selected from nitrogen, oxygen and sulphur;
iv. a straight or branched saturated or unsaturated alkyl residue of 1-22 carbon atoms comprising a saturated or unsaturated aromatic or non-aromatic homo- or heterocyclic substituent of 3-22 carbon atoms and 0-5 hetero atoms selected from nitrogen, oxygen and sulphur;
v. an aromatic or non-aromatic homo- or heterocyclic residue of 3-22 carbon atoms and 0-5 heteroatoms selected from nitrogen, oxygen and sulphur, comprising a straight or branched saturated or unsaturated alkyl substituent of 1-22 carbon atoms.

6. The method according to claim 1, wherein the compound is an inositol triphosphate, preferably an inositol triphosphate chosen from myo-inositol-1,2,6-trisphosphate and myo-inositol-1,2,3-trisphosphate.

7. The method according to claim 1, wherein the compound is the pentasodium salt of 1,2,6-D-myo inositol trisphosphate (Na₅H 1,2,6-D-myo-inositol trisphosphate), Mg₃ 1,2,6-D-myo-inositol trisphosphate or Ca₃ 1,2,6-D-myo-inositol trisphosphate).

8. A pharmaceutical composition comprising a sodium salt of 1,2,6-D-myo inositol trisphosphate in a dose that is pharmacologically efficient in the treatment, alleviation or prevention of a disease caused by a virus of the family Filoviridae, in particular a dose of from 10 mg to 60 mg/kg body weight, and a pharmaceutically acceptable carrier.

9. A pharmaceutical composition according to claim 8, wherein said virus is chosen from haemorrhagic viruses, such as the Ebola and Marburg viruses.

10. A pharmaceutical composition comprising according to claim 8, additionally comprising an analgesic agent.

11. A nutritional composition for treating or alleviating the symptoms of a filovirus infection, or for preventing catabolic conditions associated with filovirus infection, comprising:
an inositol triphosphate or an ester thereof, or a mono- or disaccharide having three or more phosphate radicals per saccharide moiety or an ester thereof; and
at least one nutrient selected from the group consisting of lipid emulsions, fluid sources of amino acids and carbohydrates.

12. A nutritional composition according to claim 11, wherein said virus is chosen from haemorrhagic viruses, such as the Ebola and Marburg viruses.

13. A nutritional composition according to claim 11 adapted for parenteral administration.

14. A nutritional composition according to claim 11 adapted for oral or enteral administration.

15. A nutritional composition according to claim 11, wherein intake of the nutritional supplement provides at least 5 to 80 mg per kg body weight of inositol triphosphate or an ester thereof to a patient.

16. All novel compounds, compositions, methods and uses substantially as hereinbefore described with particular reference to the examples and to the listing of embodiments of the invention.
